(19) United States
(12) Patent Application Publication
(10) Pub. No.: US 2024/0002517 A1
(43) Pub. Date: Jan. 4, 2024

(54) PROTEIN FORMULATIONS AND USES THEREOF

(71) Applicant: CSL INNOVATION PTY LTD, Melbourne VIC (AU)

(72) Inventors: Dianna Grace GOODALL, Melbourne VIC (AU); Nathan Aaron EDWARDS, Melbourne VIC (AU); Gemma NASSTA, Melbourne VIC (AU); Mouhamad RESLAN, Melbourne VIC (AU)

(21) Appl. No.: 18/037,397

(22) PCT Filed: Jul. 9, 2021
(86) PCT No.: PCT/AU2021/050736

§ 371 Date: May 17, 2023

(30) Foreign Application Priority Data
Dec. 16, 2020 (AU) 2020904684

(51) Int. Cl. C07K 16/28; A61K 47/22; A61K 47/26; A61K 47/18; A61P 37/04; A61K 9/00

(52) U.S. Cl. CPC C07K 16/2866; A61K 47/22; A61K 9/0019; A61K 47/183; A61P 37/04; A61K 47/26

(57) ABSTRACT

The present disclosure relates to protein formulations and uses thereof. In particular, the present disclosure relates to formulations comprising a protein comprising an antigen binding domain that binds to or specifically binds to granulocyte colony-stimulating factor receptor (G-CSFR).

### RELATED APPLICATION DATA

The present application claims priority from Australian Patent Application No. 2020904684 entitled “Protein formulations and uses thereof” filed on 16 Dec. 2020. The entire contents of which is hereby incorporated by reference.

### SEQUENCE LISTING

The present application is filed with a Sequence Listing in electronic form. The entire contents of the Sequence Listing are hereby incorporated by reference.

### FIELD

The present disclosure relates to protein formulations and uses thereof. In particular, the present disclosure relates to formulations comprising a protein comprising an antigen binding domain that binds to or specifically binds to granulocyte colony-stimulating factor receptor (G-CSFR).

### BACKGROUND

Granulocyte colony-stimulating factor (G-CSF) is a major regulator of granulocyte production. G-CSF is produced by bone marrow stromal cells, endothelial cells, macrophages, and fibroblasts, and production is induced by inflammatory stimuli. G-CSF acts through the G-CSF receptor (G-CSFR), which is expressed predominantly on neutrophils, but also on myeloid progenitors, endothelial cells, monocytes/macrophages, and T and B lymphocytes. Mice deficient in G-CSF or the G-CSFR exhibit marked neutropenia, demonstrating the importance of G-CSF in steady-state granulopoiesis. G-CSF increases the production and release of neutrophils, mobilizes hematopoietic stem and progenitor cells, and modulates the differentiation, lifespan, and effector functions of mature neutrophils. G-CSF may also exert effects on macrophages, including expansion of monocyte/macrophage numbers, enhancement of phagocytic function, and regulation of inflammatory cytokine and chemokine production. G-CSF has also been shown to mobilize endothelial progenitor cells and induce or promote angiogenesis.

While G-CSF is used therapeutically, e.g., to treat neutropenia and/or mobilize hematopoietic stem cells, it also has negative actions in some conditions, e.g., inflammatory conditions and/or cancer. For example, administration of G-CSF exacerbates rheumatoid arthritis (RA), murine collagen-induced arthritis (CIA) and a passive transfer model of CIA in rats. G-CSF has been found in the serum and synovial fluid of RA patients. Furthermore, interleukin (IL)-1 and tumor necrosis factor α (TNFα), which are found at increased levels in patients suffering from RA, induce the production of G-CSF by human synovial fibroblasts and chondrocytes. Mice deficient in G-CSF are resistant to the induction of acute and chronic inflammatory arthritis.

G-CSF has also been shown to play a role in multiple sclerosis (MS). For example, G-CSF enhances adhesion of an auto-reactive T cell line model of MS to extracellular matrix as effectively as interferon γ and TNFα, which are known to exacerbate MS symptoms. Moreover, G-CSF deficient mice are resistant to development of experimental autoimmune encephalomyelitis (EAE).

G-CSF and G-CSFR have also been tied to cancer, with studies showing that this signaling pathway contributes to chemotherapy resistance, growth, survival, invasiveness and metastasis of various cancers. Moreover, G-CSF has been shown to induce angiogenesis, a process important in the development of solid tumors.

Although antibodies and inhibitors against G-CSF and G-CSFR exist, there are an increasing number of challenges in formulation development for drug manufacturers. For example, there are numerous challenges associated with formulating high concentration antibody formulations (e.g., ≥25 mg/mL protein) suitable for subcutaneous administration. Formulations for subcutaneous administration typically require higher concentrations of product so as to achieve smaller injection volumes, yet increasing protein concentration often negatively impacts protein aggregation and degradation, solubility, stability, and viscosity. In addition to changes in intrinsic protein properties, manufacturing and supply chain challenges also exist including, difficulties with processing and storage to ensure that the formulated protein remains stable for long periods of time (e.g., greater than three months) and at higher temperatures (e.g., room temperature). Other challenges include optimising the rheological and syringeability properties of the final formulation. For example, viscous solutions typically require a higher injection force to administer, therefore a prolonged injection time may also be required contributing to patient pain and discomfort.

Various solutions to manufacturing high concentration antibody formulations include lyophilised formulations for reconstitution, bufferless formulations and the addition of high concentrations of salt or other additives to reduce aggregation and/or the viscosity of the formulation. However, the use of excessive amounts of such excipients, may lead to hypertonic preparations or changes in ionic strength of the formulation and related protein aggregation issues.

Thus, there is a need for formulations comprising protein therapeutics that bind to G-CSFR which are stable and suitable for administration to a subject for treating neutrophil-mediated conditions.

### SUMMARY

The present disclosure is based on the identification of a pharmaceutical formulation for a protein comprising an antigen binding domain that binds to or specifically binds to G-CSFR.

The inventors found that they can produce liquid formulations comprising high concentrations of a protein comprising an antigen binding domain that binds to G-CSFR, which remained stable, soluble, and had a viscosity suitable for injection. When the formulations were administered subcutaneously to cynomolgus monkeys, the protein was highly bioavailable, demonstrating the suitability of these formulations for therapeutic use. The formulation of the present disclosure comprises an organic acid buffer, a non-ionic surfactant, and at least one amino acid stabiliser. Notably, in producing the formulation of the present disclosure the inventors found that additional salts and/or stabilising agents were not required.

The present disclosure thus provides a liquid pharmaceutical formulation comprising a protein comprising an antigen binding domain that binds to or specifically binds to G-CSF receptor (G-CSFR), an organic acid buffer, a non-ionic surfactant and at least one amino acid stabiliser, wherein the formulation has a pH of 5.0 to 6.0.

In one example, the protein is present in the formulation at a concentration of at least 2 mg/mL. In one example, the protein is present in the formulation at a concentration of at least 5 mg/mL. In one example, the protein is present in the formulation at a concentration of at least 10 mg/mL. In one example, the protein is present in the formulation at a concentration of at least 20 mg/mL. In one example, the protein is present in the formulation at a concentration of at least 30 mg/mL. In one example, the protein is present in the formulation at a concentration of at least 40 mg/mL. In one example, the protein is present in the formulation at a concentration of at least 50 mg/mL. In one example, the protein is present in the formulation at a concentration of at least 60 mg/mL. In one example, the protein is present in the formulation at a concentration of at least 70 mg/mL. In one example, the protein is present in the formulation at a concentration of at least 80 mg/mL. In one example, the protein is present in the formulation at a concentration of at least 90 mg/mL. In one example, the protein is present in the formulation at a concentration of at least 100 mg/mL. In one example, the protein is present in the formulation at a concentration of at least 110 mg/mL. In one example, the protein is present in the formulation at a concentration of at least 120 mg/mL.

In one example, the protein is present in the formulation at a concentration of at least 25 mg/mL, at least 50 mg/mL or at least 100 mg/mL.

In one example, the protein is present in the formulation at a concentration in the range of 20 to 200 mg/mL. In one example, the protein is present in the formulation at a concentration in the range of 50 to 150 mg/mL. In one example, the protein is present in the formulation at a concentration in the range of 80 to 140 mg/mL.

In one example, the protein is present in the formulation at a concentration of 110 to 130 mg/mL. In one example, the protein is present in the formulation at a concentration of about 120 mg/mL.

In one example, the protein comprises an antigen binding domain of an antibody. For instance, in some examples, the protein comprises at least a heavy chain variable region (V_{H}) and a light chain variable region (V_{L}), wherein the V_{H} and V_{L} bind to form a Fv comprising an antigen binding domain. In some examples, the protein comprises a Fv. In some examples, the protein comprises:
(i) a single chain Fv fragment (scFv);
(ii) a dimeric scFv (di-scFv); or
(iii) a diabody;
(iv) a triabody;
(v) a tetrabody;
(vi) a Fab;
(vii) a F(ab′)₂;
(viii) a Fv;
(ix) one of (i) to (viii) linked to a constant region of an antibody, Fc or a heavy chain constant domain (C_{H}) 2 and/or C_{H}3;
(x) one of (i) to (viii) linked to albumin or a functional fragment or variants thereof or a protein that binds to albumin; or
(xi) an antibody.

In some examples, the protein is selected from the group consisting of:
(i) a single chain Fv fragment (scFv);
(ii) a dimeric scFv (di-scFv); or
(iii) a diabody;
(iv) a triabody;
(v) a tetrabody;
(vi) a Fab;
(vii) a F(ab′)2;
(viii) a Fv;
(ix) one of (i) to (viii) linked to a constant region of an antibody, Fc or a heavy chain constant domain (C_{H}) 2 and/or C_{H}3;
(x) one of (i) to (viii) linked to albumin, functional fragments or variants thereof or a protein (e.g., antibody or antigen binding fragment thereof) that binds to albumin; and
(xi) an antibody.

In one example, the protein comprises an Fc region.

In one example, the protein comprises one or more amino acid substitutions that increase the half-life of the protein. In one example, the antibody comprises a Fc region comprising one or more amino acid substitutions that increase the affinity of the Fc region for the neonatal Fc receptor (FcRn).

In one example, the protein is an antibody. Exemplary antibodies are described in WO2012/171057.

In one example, the protein binds to hG-CSFR expressed on the surface of a cell at an affinity of at least about 5 nM. In one example, the protein binds to hG-CSFR expressed on the surface of a cell at an affinity of at least about 4 nM. In one example, the protein binds to hG-CSFR expressed on the surface of a cell at an affinity of at least about 3 nM. In one example, the protein binds to hG-CSFR expressed on the surface of a cell at an affinity of at least about 2 nM. In one example, the protein binds to hG-CSFR expressed on the surface of a cell at an affinity of at least about 1 nM.

In some examples, the protein inhibits granulocyte colony stimulating factor (G-CSF) signalling.

In one example, the protein inhibits G-CSF-induced proliferation of a BaF3 cell expressing hG-CSFR with an IC50 of at least about 5 nM. In one example, the protein inhibits G-CSF-induced proliferation of a BaF3 cell expressing hG-CSFR with an IC50 of at least about 4 nM. In one example, the protein inhibits G-CSF-induced proliferation of a BaF3 cell expressing hG-CSFR with an IC50 of at least about 3 nM. In one example, the protein inhibits G-CSF-induced proliferation of a BaF3 cell expressing hG-CSFR with an IC50 of at least about 2 nM. In one example, the protein inhibits G-CSF-induced proliferation of a BaF3 cell expressing hG-CSFR with an IC50 of at least about 1 nM. In one example, the protein inhibits G-CSF-induced proliferation of a BaF3 cell expressing hG-CSFR with an IC50 of at least about 0.5 nM.

In one example, the protein or antibody is chimeric, de-immunized, humanized, human or primatized. In one example, the protein or antibody is human.

In one example, the protein comprises an antibody variable region that competitively inhibits the binding of antibody C1.2G comprising a heavy chain variable region (V_{H}) comprising a sequence set forth in SEQ ID NO: 4 and a light chain variable region (V_{L}) comprising a sequence set forth in SEQ ID NO: 5 to G-CSFR. In one example, the protein binds to an epitope comprising residues within one or two or three or four regions selected from 111-115, 170-176, 218-234 and/or 286-300 of SEQ ID NO: 1.

In one example, the protein comprises a V_{H} and a V_{L}, wherein:
(i) the V_{H} comprises a CDR1 comprising a sequence set forth in SEQ ID NO: 6, a CDR2 comprising a sequence set forth in SEQ ID NO: 7 and a CDR3 comprising a sequence LGELGX₁X₂X₃X₄ (SEQ ID NO: 12), wherein:
   X₁ is selected from the group consisting of tryptophan, glutamine, methionine, serine, phenylalanine, glutamic acid and histidine;
   X₂ is an amino acid selected from the group consisting of phenylalanine, tyrosine, methionine, serine, glycine and isoleucine;
   X₃ is an amino acid selected from the group consisting of aspartic acid, methionine, glutamine, serine, leucine, valine, arginine and histidine; and
   X₄ is any amino acid or an amino acid selected from the group consisting of proline, glutamic acid, alanine, leucine, phenylalanine, tyrosine, threonine, asparagine, aspartic acid, serine, glycine, arginine, and lysine; and/or
(ii) the V_{L} comprises a CDR1 comprising a sequence set forth in SEQ ID NO: 9, a CDR2 comprising a sequence set forth in SEQ ID NO: 10 and CDR3 comprising a sequence X₁X₂X₃X₄X₅X₆X₇X₈X₉ (SEQ ID NO: 13), wherein:
   X₁ is an amino acid selected from the group consisting of glutamine, glutamic acid, histidine, alanine and serine;
   X₂ is an amino acid selected from the group consisting of glutamine, valine, phenylalanine, asparagine and glutamic acid;
   X₃ is an amino acid selected from the group consisting of serine and glycine;
   X₄ is an amino acid selected from the group consisting of tryptophan, methionine, phenylalanine, tyrosine, isoleucine and leucine;
   X₅ is an amino acid selected from the group consisting of glutamic acid, methionine, glutamine, tryptophan, serine, valine, asparagine, glycine, alanine, arganine, histidine, tyrosine, lysine and threonine;
   X₆ is an amino acid selected from the group consisting of tyrosine, methionine, isoleucine and threonine;
   X₇ is an amino acid selected from the group consisting of proline, alanine, histidine, glycine and lysine;
   X₈ is an amino acid selected from the group consisting of leucine, glutamine, methionine, alanine, phenylalanine, isoleucine, lysine, histidine and glycine; and
   X₉ is an amino acid selected from the group consisting of threonine, phenylalanine, tyrosine, methionine, lysine, serine, histidine, proline, tryptophan, isoleucine, glutamine, glycine and valine.

In one example, the protein comprises an antigen binding site of an antibody, wherein:
(i) the protein binds to human granulocyte-colony stimulating factor receptor (hG-CSFR) and neutralizes granulocyte colony stimulating factor (G-CSF) signaling; and
(ii) the protein binds to a polypeptide of SEQ ID NO: 1 in which an alanine is substituted for the histidine at position 237 of SEQ ID NO:1 at a level at least 20 fold lower than it binds to a polypeptide of SEQ ID NO: 1; and
(iii) the protein binds to a polypeptide of SEQ ID NO: 1 in which an alanine is substituted for the methionine at position 198 of SEQ ID NO:1 at a level at least 20 fold lower than it binds to a polypeptide of SEQ ID NO: 1; and
(iv) the protein binds to a polypeptide of SEQ ID NO: 1 in which an alanine is substituted for the tyrosine at position 172 of SEQ ID NO:1 at a level at least 20 fold lower than it binds to a polypeptide of SEQ ID NO: 1; and
(v) the protein binds to a polypeptide of SEQ ID NO: 1 in which an alanine is substituted for the leucine at position 171 of SEQ ID NO:1 at a level at least 100 fold lower than it binds to a polypeptide of SEQ ID NO: 1; and
(vi) the protein binds to a polypeptide of SEQ ID NO: 1 in which an alanine is substituted for the leucine at position 111 of SEQ ID NO:1 at a level at least 20 fold lower than it binds to a polypeptide of SEQ ID NO: 1; and;
(vii) the protein binds to a polypeptide of SEQ ID NO: 1 in which an alanine is substituted for the histidine at position 168 of SEQ ID NO:1 at a level no more than 5 fold lower than it binds to a polypeptide of SEQ ID NO: 1; and
(viii) the protein binds to a polypeptide of SEQ ID NO: 1 in which an alanine is substituted for the lysine at position 167 of SEQ ID NO:1 at a level no more than 5 fold lower than it binds to a polypeptide of SEQ ID NO: 1; and
(ix) the antigen binding site does not detectably bind to the polypeptide of SEQ ID NO:1 in which an alanine is substituted for the arginine at position 287 of SEQ ID NO: 1; and
(x) the protein binds to a conformational epitope in the hG-CSFR; and
(xi) the protein inhibits G-CSF-induced proliferation of a BaF3 cell expressing hG-CSFR with an IC₅₀ of at least 1 nM, wherein the IC₅₀ is determined by culturing 2×10⁴ BaF3 cells in the presence of 0.5 ng/ml of hG-CSF for 48 hours, and wherein the proliferation of the BaF3 cells is determined by measuring 3-(4,5-Dimethylthiazol-2-yl)-2,5-diphenyltetrazolium bromide (MTT) reduction.

In one example, the protein comprises an antigen binding site of an antibody, wherein the antigen binding site of the protein binds to human granulocyte-colony stimulating factor receptor (hG-CSFR) and neutralizes granulocyte-colony stimulating factor (G-CSF) signaling, and wherein the protein competitively inhibits the binding of monoclonal antibody C1.2 or monoclonal antibody C1.2G to one or more of:
(i) a polypeptide of SEQ ID NO: 1 in which an alanine is substituted for the lysine at position 167 of SEQ ID NO: 1; and/or
(ii) a polypeptide of SEQ ID NO: 1 in which an alanine is substituted for the histidine at position 168 of SEQ ID NO: 1,
   wherein C1.2 comprises a V_{H} comprising a sequence set forth in SEQ ID NO: 2 and a V_{L} comprising a sequence set forth in SEQ ID NO: 3, and C1.2G comprises a V_{H} comprising a sequence set forth in SEQ ID NO: 4 and a V_{L} comprising a sequence set forth in SEQ ID NO: 5, wherein the antigen binding site of the protein also binds to the polypeptide at (i) and/or (ii), and wherein the level of binding of the protein to a polypeptide of SEQ ID NO: 1 in which an alanine is substituted for any one of:
(a) the arginine at position 287 of SEQ ID NO:1;
(b) the histidine at position 237 of SEQ ID NO:1;
(c) the methionine at position 198 of SEQ ID NO:1;
(d) the tyrosine at position 172 of SEQ ID NO:1;
(e) the leucine at position 171 of SEQ ID NO:1; or
(f) the leucine at position 111 of SEQ ID NO:1
   is lower than the level of binding of the protein to a polypeptide of SEQ ID NO: 1; and wherein the protein comprises a V_{H} and a V_{L}, wherein:
(i) the V_{H} comprises a CDR1 comprising a sequence set forth in SEQ ID NO: 6, a CDR2 comprising a sequence set forth in SEQ ID NO: 7 and a CDR3 comprising a sequence LGELGX₁X₂X₃X₄ (SEQ ID NO: 12), wherein:
   X₁ is selected from the group consisting of tryptophan, glutamine, methionine, serine, phenylalanine, glutamic acid and histidine;
   X₂ is an amino acid selected from the group consisting of phenylalanine, tyrosine, methionine, serine, glycine and isoleucine;
   X₃ is an amino acid selected from the group consisting of aspartic acid, methionine, glutamine, serine, leucine, valine, arginine and histidine; and
   X₄ is any amino acid or an amino acid selected from the group consisting of proline, glutamic acid, alanine, leucine, phenylalanine, tyrosine, threonine, asparagine, aspartic acid, serine, glycine, arginine, and lysine; and/or
(ii) the V_{L}, comprises a CDR1 comprising a sequence set forth in SEQ ID NO: 9, a CDR2 comprising a sequence set forth in SEQ ID NO: 10 and CDR3 comprising a sequence X₁X₂X₃X₄X₅X₆X₇X₈X₉ (SEQ ID NO: 13), wherein:
   X₁ is an amino acid selected from the group consisting of glutamine, glutamic acid, histidine, alanine and serine;
   X₂ is an amino acid selected from the group consisting of glutamine, valine, phenylalanine, asparagine and glutamic acid;
   X₃ is an amino acid selected from the group consisting of serine and glycine;
   X₄ is an amino acid selected from the group consisting of tryptophan, methionine, phenylalanine, tyrosine, isoleucine and leucine;
   X₅ is an amino acid selected from the group consisting of glutamic acid, methionine, glutamine, tryptophan, serine, valine, asparagine, glycine, alanine, arganine, histidine, tyrosine, lysine and threonine;
   X₆ is an amino acid selected from the group consisting of tyrosine, methionine, isoleucine and threonine;
   X₇ is an amino acid selected from the group consisting of proline, alanine, histidine, glycine and lysine;
   X₈ is an amino acid selected from the group consisting of leucine, glutamine, methionine, alanine, phenylalanine, isoleucine, lysine, histidine and glycine; and
   X₉ is an amino acid selected from the group consisting of threonine, phenylalanine, tyrosine, methionine, lysine, serine, histidine, proline, tryptophan, isoleucine, glutamine, glycine and valine.

In one example, the protein comprises an antibody variable region comprising a heavy chain variable region (V_{H}) comprising an amino acid sequence which is at least 70%, at least 80%, at least 90%, or at least 95% identical to SEQ ID NO: 4 and a light chain variable region (V_{L}) comprising an amino acid sequence which is at least 70%, at least 80%, at least 90%, or at least 95% identical to SEQ ID NO: 5.

In one example, the protein comprises an antibody variable region comprising a VH comprising an amino acid sequence set forth in SEQ ID NO: 4 and a VL comprising an amino acid sequence set forth in SEQ ID NO: 5.

In one example, the protein comprises an antibody variable region comprising a VH comprising an amino acid sequence which is at least 70%, at least 80%, at least 90%, or at least 95% identical to SEQ ID NO: 2 and a VL comprising an amino acid sequence which is at least 70%, at least 80%, at least 90%, or at least 95% identical to SEQ ID NO: 3.

In one example, the protein comprises an antibody variable region comprising a VH comprising an amino acid sequence set forth in SEQ ID NO: 2 and a VL comprising an amino acid sequence set forth in SEQ ID NO: 3.

In one example, the protein comprises an antibody variable region comprising a VH comprising three CDRs of a VH comprising an amino acid sequence set forth in SEQ ID NO: 4 and a VL comprising three CDRs of a VL comprising an amino acid sequence set forth in SEQ ID NO: 5.

In one example, the protein comprises an antibody variable region comprising a VH comprising three CDRs of a VH comprising an amino acid sequence set forth in SEQ ID NO: 2 and a VL comprising three CDRs of a VL comprising an amino acid sequence set forth in SEQ ID NO: 3.

In one example, the protein comprises:
(i) a heavy chain comprising an amino acid sequence set forth in SEQ ID NO: 14 and a light chain comprising a sequence set forth in SEQ ID NO: 15; or
(ii) a heavy chain comprising an amino acid sequence set forth in SEQ ID NO: 16 and a light chain comprising a sequence set forth in SEQ ID NO: 15.

In one example, the protein comprises:
(i) a heavy chain comprising a sequence set forth in SEQ ID NO: 14 or 18 and a light chain comprising an amino acid sequence set forth in SEQ ID NO: 15; or
(ii) one heavy chain comprising an amino acid sequence set forth in SEQ ID NO: 14 and one heavy chain comprising an amino acid sequence set forth in SEQ ID NO: 18 and two light chains comprising an amino acid sequence set forth in SEQ ID NO: 15.

In one example, the protein is a monoclonal antibody.

In one example, the antibody is an IgG antibody. For example, the antibody is an IgG₁, or an IgG₂, or an IgG₃, or an IgG₄ antibody.

In one example, the antibody is an IgG₄ antibody.

In one example, the antibody is a monoclonal IgG₄ antibody.

In one example, the protein comprises an Fc region. For example, the Fc region is a human IgG₁ Fc region or a human IgG₄ Fc region or a stabilised human IgG₄ Fc region. For example, the Fc region is a human IgG₄ Fc region. In one example, the antibody Fc region is modified to prevent dimerisation, (e.g., as discussed herein).

In one example, the antibody or antigen binding fragment thereof comprises an IgG₄ constant region.

In one example, the IgG₄ constant region is a stabilised IgG₄ constant region. For example, the IgG₄ constant region comprises a stabilised hinge region. For example, the stabilised IgG₄ constant regions comprise a proline at position 241 of the hinge region according to the system of Kabat (Kabat et al., Sequences of Proteins of Immunological Interest Washington DC United States Department of Health and Human Services, 1987 and/or 1991).

In some examples, the protein is a fusion protein. Thus, in some examples, the protein comprises an antigen binding site which binds to G-CSF or G-CSFR and comprises another amino acid sequence.

In some examples, the fusion protein comprises
a) serum albumin or a variant thereof; or
b) a soluble complement receptor or a variant thereof.

Exemplary amino acid sequences for serum albumin and variants thereof are provided in WO2019/075519. Exemplary amino acid sequences for soluble complement receptors and variants thereof are provided in WO2019/075519 and WO2019/218009.

In some examples, the soluble complement receptor is a soluble complement receptor type 1 (sCR1).

In some examples, the fusion protein comprises a complement inhibitor. In some examples, the complement inhibitor is a complement component 1 (C1) inhibitor. In one example, the C1 inhibitor is C1-INH (also known as “C1 esterase inhibitor”) or a functional variant or fragment thereof.

In some examples, the protein comprises an antigen binding site that binds to G-CSF or G-CSFR and another antigen binding site that binds to a different antigen. Thus, in some examples, the protein is a multispecific protein (e.g., a multispecific antibody). In some examples, the protein is a bispecific protein. In other examples, the protein is monospecific.

In some examples, the other antigen binding site binds to an interleukin or a receptor thereof. In some examples, the other antigen binding site binds to a complement protein.

In some examples, the other antigen binding site binds to interleukin 6 (IL-6) or IL-6 receptor (IL-6R). In some examples, the other antigen binding site binds to interleukin 3 (IL-3) or IL-3 receptor (IL-3R). In some examples, the other antigen binding site binds to interleukin 5 (IL-5) or IL-5 receptor (IL-5R). In some examples, the other antigen binding site binds to interleukin 4 (IL-4) or IL-4 receptor (IL-4R). In some examples, the other antigen binding site binds to interleukin 13 (IL-13) or IL-13 receptor (IL-13R). In some examples, the other antigen binding site binds to granulocyte-macrophage colony-stimulating factor (GM-CSF) or GM-CSF receptor (GM-CSFR). In some examples, the other antigen binding site binds to cytokine receptor common subunit beta (CSF2RB). In some examples, the other antigen binding site binds to C1. In some examples, the other antigen binding site binds to complement component 2 (C2). In some examples, the other antigen binding site binds to a blood coagulation factor. In some examples, the other antigen binding site binds to coagulation factor XII (FXII).

In one example, the organic acid buffer is selected from the group consisting of a histidine buffer, a glutamate buffer, a succinate buffer and a citrate buffer. In one example, the organic acid buffer is selected from the group consisting of a histidine buffer and a glutamate buffer.

In one example, the organic acid buffer is an amino acid buffer. For example, the amino acid buffer is selected from the group consisting of a histidine buffer and a glutamate buffer.

Advantageously, histidine buffer and glutamate buffer have higher thermal and aggregation stability (i.e., reduced propensity towards aggregation) compared to citrate buffer and/or succinate buffer.

In one example, the organic acid buffer is a histidine buffer. Suitable histidine buffers for use in the present disclosure will be apparent to the skilled person and included, for example, histidine chloride, histidine acetate, histidine phosphate and histidine sulfate. In one example, the histidine buffer is L-histidine.

In one example, the organic acid buffer is a glutamate buffer. Suitable glutamate buffers for use in the present disclosure will be apparent to the skilled person and include, for example, monosodium glutamate.

In one example, the organic acid buffer is a succinate buffer. Suitable succinate buffers for use in the present disclosure will be apparent to the skilled person and include, for example, succinic acid-monosodium succinate mixture, succinic acid-sodium hydroxide mixture, succinic acid-disodium succinate mixture.

In one example, the organic acid buffer is a citrate buffer. Suitable citrate buffers for use in the present disclosure will be apparent to the skilled person and include, for example, monosodium citrate-disodium citrate mixture, citric acid-trisodium citrate mixture, citric acid monosodium citrate mixture.

It will be apparent to the skilled person that buffers suitable for use in the present disclosure will provide sufficient buffer capacity to maintain the desired pH over the range of conditions to which it will be exposed during formulation and storage of the product. In one example, the formulation of the present disclosure has a pH of about 5.0 to about 6.0. In some examples, the formulation has a pH of about 5.2 to 5.9, or a pH of about 5.4 to about 5.9, or a pH of about 5.5 to about 5.9. In one example, the formulation has a pH of about 5.5, or about 5.6, or about 5.7, or about 5.8, or about 5.9, or about 6.0. In one example, the formulation has a pH of about 5.7. In another example, the formulation has a pH of about 5.6.

In one example, the organic acid buffer is a histidine buffer and the formulation has a pH of about 5.5 to about 5.9.

In one example, the concentration of the organic acid buffer in the pharmaceutical formulation of the present disclosure is between about 2 mM and 120 mM. In one example, the organic acid buffer is present at a concentration of a least 2 mM. For example, the organic acid buffer is present at a concentration of between about 2 mM and about 10 mM. For example, the organic acid buffer is present at a concentration of about 2 mM, or about 3 mM, or about 4 mM, or about 5 mM, or about 6 mM, or about 7 mM, or about 8 mM, or about 9 mM, or about 10 mM. In one example, the organic acid buffer is present at a concentration of at least about 10 mM. For example, the organic acid buffer is present at a concentration of between about 10 mM and about 30 mM. For example, the organic acid buffer is present at a concentration of about 10 mM, or about 12 mM, or about 14 mM, or about 16 mM, or about 18 mM, or about 20 mM, or about mM, or about 30 mM. In one example, the organic acid buffer is present at a concentration of between about 12 mM and about 25 mM. For example, the organic acid buffer is present at a concentration of about 20 mM. For example, the organic acid buffer is present at a concentration of between about 10 mM and about 60 mM. For example, the organic acid buffer is present at a concentration of about 10 mM, or about 15 mM, or about 20 mM, or about 25 mM, or about 30 mM, or about 35 mM, or about 40 mM, or about 45 mM, or about 50 mM, or about 55 mM, or about 60 mM. In one example, the organic acid buffer is present at a concentration of about 20 mM.

In one example, the organic acid buffer is present in the formulation at a concentration of 10 to 30 mM.

In one example, the organic acid buffer is histidine and is present at a concentration of about 12 mM to about 25 mM. In one example, the organic buffer is histidine and is present at a concentration of about 20 mM.

In one example, the non-ionic surfactant is selected from the group consisting of polyoxyethylensorbitan fatty acid esters (e.g., polysorbate 20 and polysorbate 80), polyethylene-polypropylene copolymers, polyethylene-polypropylene glycols, polyoxyethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS). For example, the non-ionic surfactant is selected form the group consisting of polyoxyethylensorbitan fatty acid esters and polyoxyethylene-polyoxypropylene copolymers.

In one example, the non-ionic surfactant is selected from the group consisting of polysorbate 20, polysorbate 80 and poloxamer 188.

In one example, the non-ionic surfactant is polysorbate 80.

In one example, the concentration of the non-ionic surfactant in the pharmaceutical formulation of the present disclosure is between about 0.01% (w/v) and about 1.00% (w/v). In one example, the non-ionic surfactant is present at a concentration of at least about 0.01% (w/v) or at least about 0.02% (w/v). For example, the non-ionic surfactant is present at a concentration of between about 0.01% (w/v) and about 0.10% (w/v). For example, the non-ionic surfactant is present at a concentration of about 0.01% (w/v), or about 0.02% (w/v), or about 0.03% (w/v), or about 0.04% (w/v), or about 0.05% (w/v), or about 0.06% (w/v), or about 0.07% (w/v), or about 0.08% (w/v), or about 0.09% (w/v), or about 0.10% (w/v). In one example, the non-ionic surfactant is present at a concentration of about 0.02% (w/v) or about 0.05% (w/v).

In one example, the non-ionic surfactant is present in the formulation at a concentration of 0.01% (w/v) to 0.05 (w/v). For example, the non-ionic surfactant is present at a concentration of about 0.03% (w/v).

In one example, the non-ionic surfactant is polysorbate 80 and is present at a concentration of between about 0.01% (w/v) and about 0.05% (w/v). In one example, the non-ionic surfactant is polysorbate 80 and is present at a concentration of about (w/v).

In one example, the pharmaceutical formulation comprises at least one amino acid stabiliser selected from the group consisting of proline, arginine, glycine, alanine, valine, leucine, isoleucine, methionine, threonine, phenylalanine, tyrosine, serine, cysteine, histidine, tryptophan, aspartic acid, glutamic acid, lysine, omithine and asparagine. For example, the amino acid stabiliser is selected from the group consisting of proline, arginine, salts thereof and a combination thereof. In one example, the amino acid stabiliser is a salt form of an amino acid discussed herein.

In one example, the at least one amino acid stabiliser includes proline and/or arginine.

In one example, the at least one amino acid stabiliser includes proline. In one example, the at least one amino acid stabiliser includes L-proline.

In one example, the at least one amino acid stabiliser includes arginine. In one example, the at least one amino acid stabiliser includes L-arginine. In one example, the at least one amino acid stabiliser includes L-arginine monohydrochloride.

In one example, the formulation comprises proline and arginine. For example, the formulation comprises L-proline and L-arginine or L-arginine monohydrochloride.

Advantageously, proline has a significant effect on thermal and aggregation stability (i.e., reduced propensity towards aggregation) compared to phenylalanine, arginine and sorbitol.

In one example, the concentration of the amino acid stabiliser in the pharmaceutical formulation of the present disclosure is between about 25 mM and about 200 mM. In one example, the amino acid stabiliser is present at a concentration of between about 50 mM and about 150 mM. For example, the amino acid stabiliser is present at a concentration of about 50 mM, or about 60 mM, or about 70 mM, or about 80 mM, or about 90 mM, or about 100 mM, or about 110 mM, or about 120 mM, or about 130 mM, or about 140 mM, or about 150 mM. In another example, the amino acid stabiliser is present at a concentration of between about 75 mM and about 125 mM. In another example, the amino acid stabiliser is present at a concentration of between about 90 mM and about 110 mM. For example, the amino acid stabiliser is present at a concentration of about 100 mM. In some examples, the formulation comprises two or more amino acid stabilisers, each present at concentration provided above.

Discussion of the foregoing concentrations also relates to a salt form of the amino acid stabiliser and the concentration recited herein is the concentration of the salt form of the amino acid rather the concentration of the amino acid per se.

In one example, the formulation comprises proline at a concentration of between 50 mM and 150 mM or between 75 mM and 125 mM or between 90 and 110 mM, for example at a concentration of about 100 mM. In some examples, the concentration of proline is less than 140 mM or less than 130 mM or less than 120 mM.

In one example, the at least one amino acid stabiliser includes proline, wherein proline is present in the formulation at a concentration of 50 mM to 150 mM

In one example, the formulation comprises arginine at a concentration of between 50 mM and 150 mM, or between 75 mM and 125 mM, or between 90 and 110 mM, for example at a concentration of about 100 mM. In some examples, the concentration of arginine is less than 150 mM or less than 140 mM or less than 130 mM or less than 120 mM. In one example, the arginine is a salt form of arginine, e.g., arginine monohydrochloride and the concentration recited herein is the concentration of the salt form of arginine rather the concentration of arginine per se.

In one example, the at least one amino acid stabiliser includes arginine, wherein arginine is present in the formulation at a concentration of 50 mM to 150 mM.

In one example, the formulation comprises proline at a concentration of between mM and 150 mM and arginine at a concentration of between 50 mM and 150 mM. For example, the formulation comprises about 100 mM L-proline and about 100 mM L-arginine.

In some examples, the formulation comprises a histidine buffer, proline and polysorbate 80. In some examples, the formulation further comprises arginine. In some examples, the formulation does not comprise any amino acid other than histidine, proline and arginine.

In one example, the formulation does not comprise a salt. In some examples, the formulation lacks a tonicifying amount of a salt. In some examples, the formulation does not comprise a metal salt. In some examples, the formulation does not comprise, for example, sodium chloride, calcium chloride and/or potassium chloride. Discussion of the foregoing salt does not relate to a salt form of an amino acid, or other component, in the formulation disclosed herein.

In one example, the formulation does not comprise a polyol. In one example, the formulation does not comprise a sugar, a sugar alcohol or a saccharic acid.

In one example, the formulation has a dynamic (i.e., absolute) viscosity of less than about 30 mPa*s at 20° C. In one example, the formulation has a dynamic (i.e., absolute) viscosity of less than about 20 mPa*s at 20° C. In one example, the formulation has a dynamic viscosity of less than about 15 mPa*s at 20° C. In one example, the formulation has a dynamic viscosity of less than about 10 mPa*s at 20° C. In one example, the formulation has a dynamic viscosity of between about 4.0 mPa*s and about 7.0 mPa*s at 20° C. For example, the formulation has a dynamic viscosity of about 5.4 mPa*s at 20° C.

In one example, the formulation has a dynamic viscosity of less than 20 mPa*s at less than 10 mPa*s at 20° C., or less than 7 mPa*s at 20° C.

In one example, the formulation has a dynamic (i.e., absolute) viscosity of less than about 30 mPa*s at 25° C. In one example, the formulation has a dynamic (i.e., absolute) viscosity of less than about 20 mPa*s at 25° C. In one example, the formulation has a dynamic viscosity of less than about 15 mPa*s at 25° C. In one example, the formulation has a dynamic viscosity of less than about 10 mPa*s at 25° C. In one example, the formulation has a dynamic viscosity of between about 3.0 mPa*s and about 6.0 mPa*s at 25° C. For example, the formulation has a dynamic viscosity of about 4.6 mPa*s at 25° C.

Methods of assessing viscosity will be apparent to the skilled person and/or are described herein. For example, viscosity may be assessed by use of a microviscometer, such as a rolling-ball viscometer. A rolling-ball viscometer measures the rolling time of a ball through transparent and opaque liquids according to Hoppler's falling ball principle. An example of a rolling-ball viscometer is the Anton Par Lovis 2000 M Microviscometer.

In one example, the osmolality of the formulation is between about 150 mOsm/kg and about 550 mOsm/kg. For example, the osmolality of the formulation is about 150 mOsm/kg, or about 175 mOsm/kg, or about 200 mOsm/kg, or about 225 mOsm/kg, or about 250 mOsm/kg, or about 275 mOsm/kg, or about 300 mOsm/kg, or about 325 mOsm/kg, or about 350 mOsm/kg, or about 375 mOsm/kg, or about 400 mOsm/kg, or about 425 mOsm/kg, or about 450 mOsm/kg, or about 475 mOsm/kg, or about 500 m Osm/kg, or about 550 mOsm/kg. In one example, the osmolality of the formulation is between about 250 mOsm/kg and about 400 mOsm/kg. For example, the osmolality of the formulation is about 250 mOsm/kg, or about 260 mOsm/kg, or about 270 mOsm/kg, or about 280 mOsm/kg, or about 290 mOsm/kg, or about 300 mOsm/kg, or about 310 mOsm/kg, or about 320 mOsm/kg, or about 330 mOsm/kg, or about 340 mOsm/kg, or about 350 mOsm/kg, or about 360 mOsm/kg, or about 370 mOsm/kg, or about 380 mOsm/kg, or about 390 mOsm/kg, or about 400 mOsm/kg. In one example, the osmolality is between about 280 mOsm/kg and about 350 mOsm/kg. For example, the osmolality is about 315 mOsm/kg.

In some examples, the formulation is a stable formulation. The stability of the formulation may be assessed by any means known in the art. For example, the stability of the formulation may be assessed by measuring total high molecular weight species (HMWS) and/or monomer content. Methods for assessing accumulation of HMWS and monomer content of the formulation will be apparent to the skilled person and/or described herein. In one example, the percent HMWS of the protein in the formulation is determined by size-exclusion chromatography (e.g., SEC or SE-HPLC).

In another example, the formation of HMWS of the protein is assessed using dynamic light scattering (DLS). For example, the fluctuation of light intensity using a digital correlator (e.g., Malvern Zetasizer software) is measured and the Z-average hydrodynamic diameter and polydispersity index (using e.g., a cumulants analysis) are determined.

In some examples, the formulation comprises no more than 5% high molecular weight species (HMWS). In some examples, the formulation comprises no more than 5% HMWS, as determined by size exclusion chromatography (SEC). In some examples, the formulation comprises no more than 5% HMWS, as determined by size exclusion high performance liquid chromatography (SE-HPLC).

In some examples, the formulation of the present disclosure comprises at least 90% monomer protein and/or less than (i.e., no more than) 10% HMWS and/or low molecular weight species (LMWS, i.e., degraded or fragmented). In one example, the formulation comprises at least 95% monomer protein and/or less than (i.e., no more than) 5% HMWS and/or LMWS.

In one example, the formulation comprises no more than about 10% HMWS. For example, the formulation comprises no more than about 10%, or no more than about 9%, or no more than about 8%, or no more than about 7%, or no more than about 6%, or no more than about 5%, or no more than about 4%, or no more than about 3%, or no more than about 2%, or no more than about 1% HMWS.

In some examples, the formulation comprises no more than 5% high molecular weight species (HMWS) after storage for a period of at least 1 month, at least 3 months, at least 6 months, at least 9 months, or at least 12 months at a temperature in the range of 2° C. to 30° C. In some examples, the formulation comprises no more than 5% high molecular weight species (HMWS) after storage for a period of at least 1 month, at least 3 months, at least 6 months, at least 9 months, at least 12 months, at least 18 months, or at least 24 months at a temperature in the range of 2° C. to 30° C. In one example, the formulation comprises no more than 5% HMWS after storage for a period of at least 12 months at a temperature of about 25° C. In one example, the formulation comprises no more than 3% HMWS after storage for a period of at least 12 months at a temperature of about 5° C. In one example, the formulation comprises no more than 5% HMWS after storage for a period of at least 18 months at a temperature of about 25° C. In another example, the formulation comprises no more than 3% HMWS after storage for a period of at least 18 months at a temperature of about 5° C. In another example, the formulation comprises no more than 3% HMWS after storage for a period of at least 24 months at a temperature of about 5° C.

In some examples, at least 95% of the protein in the formulation is a monomer. In some examples, at least 95% of the protein in the formulation is a monomer, as determined by SEC. In some examples, at least 95% of the protein in the formulation is a monomer, as determined by SE-HPLC.

In some examples, at least 96% of the protein in the formulation is a monomer. In some examples, at least 96%, or at least 97%, or at least 98%, or at least 99% of the protein in the formulation is a monomer.

In some examples, at least 95% of the protein in the formulation is a monomer after storage for a period of at least 1 month, at least 3 months, at least 6 months, at least 9 months, or at least 12 months at a temperature in the range of 2° C. to 30° C. In some examples, at least 95% of the protein in the formulation is a monomer after storage for a period of at least 1 month, at least 3 months, at least 6 months, at least 9 months, at least 12 months, at least 18 months, or at least 24 months at a temperature in the range of 2° C. to 30° C. In one example, at least 95% of the protein in the formulation is a monomer after storage for a period of at least 12 months at a temperature of about 25° C. In one example, at least 97% of the protein in the formulation is a monomer after storage for a period of at least 12 months at a temperature of about 5° C. In one example, at least 95% of the protein in the formulation is a monomer after storage for a period of at least 18 months at a temperature of about 25° C. In one example, at least 97% of the protein in the formulation is a monomer after storage for a period of at least 18 months at a temperature of about 5° C. In one example, at least 97% of the protein in the formulation is a monomer after storage for a period of at least 24 months at a temperature of about 5° C.

Another method for assessing the stability of the formulation includes measuring the accumulation of acidic and/or basic species of the protein. The amount of acidic and/or basic species of a protein can be measured using cation exchange chromatography (e.g., CEX-HPLC), for example.

In some examples, the formulation comprises no more than 35% acidic species. In some examples, the formulation comprises no more than 35% acidic species, as determined by cation exchange chromatography. In some examples, the formulation comprises no more than 35% acidic species, as determined by cation exchange high performance liquid chromatography (CEX-HPLC).

In some examples, the formulation comprises no more than 35%, no more than 30%, or no more than 27.5%, or no more than 25%, or no more than 22.5%, or no more than 20%, or no more than 17.5% acidic species.

In some examples, the formulation comprises no more than 35% acidic species after storage for a period of at least 1 month, at least 3 months, at least 6 months, at least 9 months, or at least 12 months at a temperature in the range of 2° C. to 30° C. In one example, the formulation comprises no more than 35% acidic species after storage for a period of at least 12 months at a temperature of about 25° C. In one example, the formulation comprises no more than 20% acidic species after storage for a period of at least 12 months at a temperature of about 5° C.

In some examples, the formulation comprises no more than 50% acidic species after storage for a period of at least 1 month, at least 3 months, at least 6 months, at least 9 months, at least 12 months, or at least 18 months, or at least 24 months at a temperature in the range of 2° C. to 30° C. In some examples, the formulation comprises no more than 50% acidic species after storage for a period of at least 18 months at a temperature in the range of 2° C. to 30° C. In one example, the formulation comprises no more than 50% acidic species after storage for a period of at least 18 months at a temperature of about 25° C. In one example, the formulation comprises no more than 20% acidic species after storage for a period of at least 18 months at a temperature of about 5° C. In one example, the formulation comprises no more than 20% acidic species after storage for a period of at least 24 months at a temperature of about 5° C.

In some examples, the formulation comprises no more than 20% basic species. In some examples, the formulation comprises no more than 20% basic species, as determined by cation exchange chromatography. In some examples, the formulation comprises no more than 20% basic species, as determined by cation exchange high performance liquid chromatography (CEX-HPLC).

In some examples, the formulation comprises no more than 20%, or no more than 19%, or no more than 18%, or no more than 17%, or no more than 16%, or no more than 15% basic species.

In some examples, the formulation comprises no more than 20% basic species after storage for a period of at least 1 month, at least 3 months, at least 6 months, at least 9 months, or at least 12 months at a temperature in the range of 2° C. to 30° C. In some examples, the formulation comprises no more than 20% basic species after storage for a period of at least 1 month, at least 3 months, at least 6 months, at least 9 months, at least 12 months, at least 18 months, or at least 24 months at a temperature in the range of 2° C. to 30° C. In one example, the formulation comprises no more than 20% basic species after storage for a period of at least 12 months at a temperature of about 25° C. In one example, the formulation comprises no more than 20% basic species after storage for a period of at least 12 months at a temperature of about 5° C. In one example, the formulation comprises no more than 20% basic species after storage for a period of at least 18 months at a temperature of about 25° C. In one example, the formulation comprises no more than 20% basic species after storage for a period of at least 18 months at a temperature of about 5° C. In one example, the formulation comprises no more than 20% basic species after storage for a period of at least 24 months at a temperature of about 5° C.

In some examples, the formulation comprises no more than 5% LMWS. In some examples, the formulation comprises no more than 5% LMWS, as determined by capillary electrophoresis with sodium dodecylsulfate (CE-SDS) under non-reducing conditions.

In some examples, the formulation comprises no more than 5%, or no more than 4%, or no more than 3%, or no more than 2%, or no more than 1% LMWS.

In some examples, the formulation comprises no more than 5% LMWS after storage for a period of at least 1 month, at least 3 months, at least 6 months, at least 9 months, or at least 12 months at a temperature in the range of 2° C. to 30° C. In some examples, the formulation comprises no more than 5% LMWS after storage for a period of at least 1 month, at least 3 months, at least 6 months, at least 9 months, at least 12 months, at least 18 months, or at least 24 months at a temperature in the range of 2° C. to ° C. In one example, the formulation comprises no more than 5% LMWS after storage for a period of at least 12 months at a temperature of about 25° C. In one example, the formulation comprises no more than 1% LMWS after storage for a period of at least 12 months at a temperature of about 5° C. In one example, the formulation comprises no more than 5% LMWS after storage for a period of at least 18 months at a temperature of about 25° C. In one example, the formulation comprises no more than 1% LMWS after storage for a period of at least 18 months at a temperature of about 5° C. In one example, the formulation comprises no more than 1% LMWS after storage for a period of at least 24 months at a temperature of about 5° C.

In some examples of the formulation of the disclosure, one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC;
c) the formulation comprises no more than 35% acidic species, as determined by cation exchange high performance liquid chromatography (CEX-HPLC);
d) the formulation comprises no more than 20% basic species, as determined by cation exchange high performance liquid chromatography (CEX-HPLC); and
e) the formulation comprises no more than 5% low molecular weight species (LMWS), as determined by capillary electrophoresis with sodium dodecylsulfate (CE-SDS) under non-reducing conditions.

In some examples, the amount of HMWS, monomer, acidic species, basic species, or LMWS described above is determined after storage for a period of at least 1 month, at least 3 months, at least 6 months, at least 9 months, or at least 12 months at a temperature in the range of 2° C. to 30° C. In one example, the amount of HMWS, monomer, acidic species, basic species, or LMWS is determined after storage for a period of at least 1 month, at least 3 months, at least 6 months, at least 9 months, or at least 12 months at a temperature in the range of 2° C. to 8° C. In another example, the amount of HMWS, monomer, acidic species, basic species, or LMWS is determined after storage for a period of at least 1 month, at least 3 months, at least 6 months, at least 9 months, or at least 12 months at a temperature in the range 22° C. to 28° C.

In some examples of the formulation of the disclosure, one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC;
c) the formulation comprises no more than 50% acidic species, as determined by cation exchange high performance liquid chromatography (CEX-HPLC);
d) the formulation comprises no more than 20% basic species, as determined by cation exchange high performance liquid chromatography (CEX-HPLC); and
e) the formulation comprises no more than 5% low molecular weight species (LMWS), as determined by capillary electrophoresis with sodium dodecylsulfate (CE-SDS) under non-reducing conditions.

In some examples, the amount of HMWS, monomer, acidic species, basic species, or LMWS described above is determined after storage for a period of at least 1 month, at least 3 months, at least 6 months, at least 9 months, at least 12 months, at least 18 months, or at least 24 months at a temperature in the range of 2° C. to 30° C. In one example, the amount of HMWS, monomer, acidic species, basic species, or LMWS is determined after storage for a period of at least 1 month, at least 3 months, at least 6 months, at least 9 months, at least 12 months, at least 18 months, or at least 24 months at a temperature in the range of 2° C. to 8° C. In another example, the amount of HMWS, monomer, acidic species, basic species, or LMWS is determined after storage for a period of at least 1 month, at least 3 months, at least 6 months, at least 9 months, at least 12 months, at least 18 months, or at least 24 months at a temperature in the range 22° C. to 28° C.

In some examples, after storage of the formulation for a period of at least 12 months at a temperature of about 25° C., one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC;
c) the formulation comprises no more than 35% acidic species, as determined by cation exchange high performance liquid chromatography (CEX-HPLC);
d) the formulation comprises no more than 20% basic species, as determined by cation exchange high performance liquid chromatography (CEX-HPLC); and
e) the formulation comprises no more than 5% low molecular weight species (LMWS), as determined by capillary electrophoresis with sodium dodecylsulfate (CE-SDS) under non-reducing conditions.

In some examples, after storage of the formulation for a period of at least 12 months at a temperature of about 5° C., one or more or all of the following apply:
a) the formulation comprises no more than 3% high molecular weight species (HMWS), as determined by size exclusion high performance liquid chromatography (SE-HPLC);
b) at least 97% of the protein in the formulation is a monomer, as determined by SE-HPLC;
c) the formulation comprises no more than 20% acidic species, as determined by cation exchange high performance liquid chromatography (CEX-HPLC);
d) the formulation comprises no more than 20% basic species, as determined by cation exchange high performance liquid chromatography (CEX-HPLC); and
e) the formulation comprises no more than 1% low molecular weight species (LMWS), as determined by capillary electrophoresis with sodium dodecylsulfate (CE-SDS) under non-reducing conditions.

In some examples, after storage of the formulation for a period of at least 18 months at a temperature of about 25° C., one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC;
c) the formulation comprises no more than 50% acidic species, as determined by cation exchange high performance liquid chromatography (CEX-HPLC);
d) the formulation comprises no more than 20% basic species, as determined by cation exchange high performance liquid chromatography (CEX-HPLC); and
e) the formulation comprises no more than 5% low molecular weight species (LMWS), as determined by capillary electrophoresis with sodium dodecylsulfate (CE-SDS) under non-reducing conditions.

In some examples, after storage of the formulation for a period of at least 24 months at a temperature of about 5° C., one or more or all of the following apply:
a) the formulation comprises no more than 3% high molecular weight species (HMWS), as determined by size exclusion high performance liquid chromatography (SE-HPLC);
b) at least 97% of the protein in the formulation is a monomer, as determined by SE-HPLC;
c) the formulation comprises no more than 20% acidic species, as determined by cation exchange high performance liquid chromatography (CEX-HPLC);
d) the formulation comprises no more than 20% basic species, as determined by cation exchange high performance liquid chromatography (CEX-HPLC); and
e) the formulation comprises no more than 1% low molecular weight species (LMWS), as determined by capillary electrophoresis with sodium dodecylsulfate (CE-SDS) under non-reducing conditions.

In some examples, the formulation is an aqueous formulation. In one example, the formulation is suitable for subcutaneous administration. In some examples, the formulation has a volume in the range of 0.2 mL to 10 mL. In some examples, the formulation has a volume in the range of 0.5 mL to 5 mL. In some examples, the formulation has a volume in the range of 1 mL to 3 mL. In some examples, the formulation has a volume of about 1 mL, or about 2 mL, or about 3 mL, or about 4 mL, or about 5 mL.

In one example, the formulation has not previously been lyophilised. In one example, the formulation is not a reconstituted formulation.

The present disclosure provides a liquid pharmaceutical formulation comprising a protein comprising an antigen binding domain that binds to or specifically binds to G-CSF receptor (G-CSFR), an organic acid buffer selected from the group consisting of a histidine and glutamate, a surfactant selected from the group consisting of polysorbate 20, polysorbate 80 and poloxamer 188, and at least one amino acid stabiliser including proline and/or arginine, wherein the formulation has a pH of 5.0 to 6.0.

The present disclosure also provides a liquid pharmaceutical formulation comprising a protein comprising an antigen binding domain that binds to or specifically binds to G-CSF receptor (G-CSFR), a histidine buffer, polysorbate 80, proline and arginine, wherein the formulation has a pH of 5.0 to 6.0.

In some examples, the formulation has a pH of 5.5 to 5.9 and comprises 5 mM to 50 mM histidine buffer, 0.01% to 0.05% (w/v) polysorbate 80, 50 mM to 150 mM proline and 50 mM to 150 mM arginine.

In some examples, the formulation has a pH of 5.5 to 5.9 and comprises 10 mM to 30 mM histidine buffer, 0.02% to 0.04% (w/v) polysorbate 80, 80 mM to 120 mM proline and 80 mM to 120 mM arginine.

In some examples, the formulation has a pH of 5.5 to 5.9 and comprises 12 mM to 25 mM histidine buffer, 0.02% to 0.04% (w/v) polysorbate 80, 60 mM to 125 mM proline and 60 mM to 125 mM arginine.

In some examples, the formulation has a pH of 5.5 to 5.9 and comprises 15 mM to 25 mM histidine buffer, 0.02% to 0.04% (w/v) polysorbate 80, 90 mM to 110 mM proline and 90 mM to 110 mM arginine.

In some examples, the present disclosure provides a liquid pharmaceutical formulation comprising a protein comprising an antigen binding domain that binds to or specifically binds to G-CSF receptor (G-CSFR), 12 mM to 25 mM histidine buffer, 0.02% to 0.04% (w/v) polysorbate 80, 60 mM to 125 mM proline and 60 mM to 125 mM arginine, wherein the formulation has a pH of 5.5 to 5.9.

In some examples, the formulation comprises 15 mM to 25 mM histidine buffer, 0.02% to 0.04% (w/v) polysorbate 80, 90 mM to 110 mM proline and 90 mM to 110 mM arginine, wherein the formulation has a pH of 5.5 to 5.9

In some examples, the formulation has a pH of 5.5 to 5.9 and comprises about 20 mM histidine buffer, about 0.03% (w/v) polysorbate 80, about 100 mM proline and about 100 mM arginine.

In some examples, the formulation has a pH of 5.7 and comprises 20 mM histidine buffer, 0.03% (w/v) polysorbate 80, 100 mM proline and 100 mM arginine.

The present disclosure also provides a liquid pharmaceutical formulation comprising a protein comprising an antigen binding domain that binds to or specifically binds to G-CSF receptor (G-CSFR), a histidine buffer, polysorbate 80, proline and arginine, wherein the formulation has a pH of 5.0 to 6.0, and wherein the protein comprises a V_{H} comprising an amino acid sequence set forth in SEQ ID NO: 4 and a V_{L} comprising an amino acid sequence set forth in SEQ ID NO: 5.

The present disclosure also provides a liquid pharmaceutical formulation comprising a protein comprising an antigen binding domain that binds to or specifically binds to G-CSF receptor (G-CSFR), 10 mM to 30 mM histidine buffer, 0.01% to 0.05% polysorbate 80, 50 mM to 150 mM proline and 50 mM to 150 mM arginine, wherein the formulation has a pH of 5.0 to 6.0, and wherein the protein comprises a V_{H} comprising an amino acid sequence set forth in SEQ ID NO: 4 and a VL comprising an amino acid sequence set forth in SEQ ID NO: 5.

The present disclosure also provides a liquid pharmaceutical formulation comprising a protein comprising an antigen binding domain that binds to or specifically binds to G-CSF receptor (G-CSFR), a histidine buffer, polysorbate 80, proline and arginine, wherein the formulation has a pH of 5.0 to 6.0, and wherein the protein comprises a V_{H} comprising three CDRs of a V_{H} comprising an amino acid sequence set forth in SEQ ID NO: 4 and a VL comprising three CDRs of a VL comprising an amino acid sequence set forth in SEQ ID NO: 5.

The present disclosure also provides a liquid pharmaceutical formulation comprising a protein comprising an antigen binding domain that binds to or specifically binds to G-CSF receptor (G-CSFR), 10 mM to 30 mM histidine buffer, 0.01% to 0.05% polysorbate 80, 50 mM to 150 mM proline and 50 mM to 150 mM arginine, wherein the formulation has a pH of 5.0 to 6.0, and wherein the protein comprises a V_{H} comprising three CDRs of a V_{H} comprising an amino acid sequence set forth in SEQ ID NO: 4 and a VL comprising three CDRs of a VL comprising an amino acid sequence set forth in SEQ ID NO: 5.

The present disclosure also provides a liquid pharmaceutical formulation comprising a protein comprising an antigen binding domain that binds to or specifically binds to G-CSF receptor (G-CSFR), a histidine buffer, polysorbate 80, proline and arginine, wherein the formulation has a pH of 5.0 to 6.0, and wherein the protein comprises:
a) a V_{H} comprising a CDR1 comprising an amino acid sequence set forth in SEQ ID NO: 6, a CDR2 comprising an amino acid sequence set forth in SEQ ID NO: 7 and a CDR3 comprising an amino acid sequence set forth in SEQ ID NO: 8; and
b) a V_{L} comprising a CDR1 comprising an amino acid sequence set forth in SEQ ID NO: 9, a CDR2 comprising an amino acid sequence set forth in SEQ ID NO: 10 and a CDR3 comprising an amino acid sequence set forth in SEQ ID NO: 11.

The present disclosure also provides a liquid pharmaceutical formulation comprising a protein comprising an antigen binding domain that binds to or specifically binds to G-CSF receptor (G-CSFR), 10 mM to 30 mM histidine buffer, 0.01% to 0.05% polysorbate 80, 50 mM to 150 mM proline and 50 mM to 150 mM arginine, wherein the formulation has a pH of 5.0 to 6.0, and wherein the protein comprises:
a) a V_{H} comprising a CDR1 comprising an amino acid sequence set forth in SEQ ID NO: 6, a CDR2 comprising an amino acid sequence set forth in SEQ ID NO: 7 and a CDR3 comprising an amino acid sequence set forth in SEQ ID NO: 8; and
b) a V_{L} comprising a CDR1 comprising an amino acid sequence set forth in SEQ ID NO: 9, a CDR2 comprising an amino acid sequence set forth in SEQ ID NO: 10 and a CDR3 comprising an amino acid sequence set forth in SEQ ID NO: 11.

The present disclosure also provides a liquid pharmaceutical formulation comprising a protein comprising an antigen binding domain that binds to or specifically binds to G-CSF receptor (G-CSFR), a histidine buffer, polysorbate 80, proline and arginine, wherein the formulation has a pH of 5.0 to 6.0, and wherein the protein is an antibody comprising a heavy chain comprising an amino acid sequence set forth in SEQ ID NO: 14 or 18 and a light chain comprising an amino acid sequence set forth in SEQ ID NO: 15.

The present disclosure also provides a liquid pharmaceutical formulation comprising a protein comprising an antigen binding domain that binds to or specifically binds to G-CSF receptor (G-CSFR), 10 mM to 30 mM histidine buffer, 0.01% to 0.05% polysorbate 80, 50 mM to 150 mM proline and 50 mM to 150 mM arginine, wherein the formulation has a pH of 5.0 to 6.0, and wherein the protein is an antibody comprising a heavy chain comprising an amino acid sequence set forth in SEQ ID NO: 14 or 18 and a light chain comprising an amino acid sequence set forth in SEQ ID NO: 15.

The present disclosure also provides a method of reducing circulating neutrophils in a subject, the method comprising administering the formulation described herein.

The present disclosure also provides a formulation described herein for use in reducing circulating neutrophils in a subject.

The present disclosure also provides use of the formulation described herein in the manufacture of a medicament for reducing circulating neutrophils in a subject.

The present disclosure also provides a method of treating or preventing a neutrophil-mediated condition in a subject, the method comprising administering the formulation described herein to the subject.

The present disclosure also provides a formulation described herein for use in treating or preventing a neutrophil-mediated condition in a subject.

The present disclosure also provides use of the formulation described herein in the manufacture of a medicament for use in treating or preventing a neutrophil-mediated condition in a subject.

In some examples, the neutrophil-mediated condition is an autoimmune disease, an inflammatory disease, cancer or ischemia-reperfusion injury.

Exemplary autoimmune conditions include autoimmune intestinal disorders (such as Crohn's disease and ulcerative colitis), arthritis (such as rheumatoid arthritis, psoriatic arthritis and or idiopathic arthritis, e.g., juvenile idiopathic arthritis) or psoriasis.

Exemplary inflammatory conditions include inflammatory neurological conditions (e.g., Devic's disease, a viral infection in the brain, multiple sclerosis and neuromyelitis optica), an inflammatory lung disease (e.g., chronic obstructive pulmonary disease [COPD], acute respiratory distress syndrome [ARDS] or asthma) or an inflammatory eye condition (e.g., uveitis).

In one example, the neutrophil-mediated condition is asthma.

In one example, the neutrophil-mediated condition is ARDS.

In one example, the neutrophil-mediated condition is ischemia-reperfusion injury. For example, the ischemia-reperfusion injury is due to or associated with tissue or organ transplantation (e.g., kidney transplantation). For example, the antibody is administered to a tissue or organ transplantation recipient, e.g., prior to organ collection and/or to a tissue or organ prior to transplantation or is administered to a harvested tissue or organ ex vivo.

In some examples, the neutrophil-mediated condition is psoriasis. In one example, the neutrophil-mediated condition is plaque psoriasis (also known in the art as “psoriasis vulgaris” or “common psoriasis”).

In one example, the neutrophil-mediated condition is a neutrophilic dermatosis or a neutrophilic skin lesion. For example, the neutrophilic dermatosis is a pustular psoriasis.

In one example, the neutrophilic dermatosis is selected from the group consisting of amicrobial pustulosis of the folds (APF); plaque psoriasis; CARD14-mediated pustular psoriasis (CAMPS); cryopyrin associated periodic syndromes (CAPS); deficiency of interleukin-1 receptor (DIRA); deficiency of interleukin-36 receptor antagonist (DIRTA); hidradenitis suppurativa (HS); palmoplantar pustulosis; pyogenic arthritis; pyoderma gangrenosum and acne (PAPA); pyoderma gangrenosum, acne, and hidradenitis suppurativa (PASH); pyoderma gangrenosum (PG); skin lesions of Behcet's disease; Still's disease; Sweet syndrome; subcorneal pustulosis (Sneddon-Wilkinson); pustular psoriasis; palmoplantar pustulosis; acute generalized exanthematic pustulosis; infantile acropustulosis; synovitis, acne, pustulosis; hyperostosis and osteitis (SAPHO) syndrome; bowel-associated dermatosis-arthritis syndrome (BADAS); neutrophilic dermatosis of the dorsal hands; neutrophilic eccrine hidradenitis; erythema elevatum diutinum; and Pyoderma gangrenosum. In one example, the neutrophilic dermatosis is hidradenitis suppurativa (HS) or palmoplantar pustulosis (PPP).

In one example, the formulation of the disclosure is administered subcutaneously to the subject in need thereof. In another example, the formulation of the disclosure is administered intravenously to the subject in need thereof.

In one example, the formulation of the disclosure is self-administered.

In one example, the formulation of the disclosure is self-administered subcutaneously.

In one example, the formulation of the disclosure is provided in a pre-filled syringe.

In one example, the formulation of the disclosure is self-administered subcutaneously, with a pre-filled syringe.

In one example of any method described herein, the subject is a mammal, for example a primate such as a human.

Methods of treatment described herein can additionally comprise administering a further compound to reduce, treat or prevent the effect of the neutrophil-mediated condition.

The present disclosure also provides a kit for use in treating or preventing a neutrophil-mediated condition in a subject, the kit comprising:
(a) at least one pharmaceutical formulation described herein;
(b) instructions for using the kit in treating or preventing the neutrophil-mediated condition in the subject; and
(c) optionally, at least one further therapeutically active compound or drug.

In some examples, the formulation is present in a vial, a prefilled syringe or an autoinjector device.

The present disclosure also provides a prefilled syringe comprising the pharmaceutical formulation described herein.

The present disclosure also provides an autoinjector device comprising the pharmaceutical formulation described herein.

Exemplary effects of the pharmaceutical formulation of the present disclosure are described herein and are to be taken to apply mutatis mutandis to the examples of the disclosure set out in the previous paragraphs.

### BRIEF DESCRIPTION OF THE FIGURES

FIG. 1 is a size exclusion chromatogram illustrating the effect of pH on aggregation of a formulation comprising 150 mg/mL CSL324, 20 mM histidine buffer (with a pH of 6.4, 6.0 or 5.5), 95 mM proline and 100 mM arginine.

FIG. 2 is a dot plot showing the amount of high molecular weight species (A) and acidic variants (B) produced after storage of CSL324 formulations at 5° C. or 25° C. over a period of 8 weeks.

FIG. 3 is a graph showing mean (+SD) concentrations (ng/mL) of CSL324 in combined male and female monkey serum following a single dose via IV or SC injection administration.

### KEY TO SEQUENCE LISTING

SEQ ID NO: 1—amino acids 25-335 of Homo sapiens G-CSFR (hG-CSFR) with a C-terminal polyhistidine tag
SEQ ID NO: 2—V_{H} of C1.2
SEQ ID NO: 3—V_{L} of C1.2
SEQ ID NO: 4—V_{H} of C1.2G
SEQ ID NO:5—V_{L} of C1.2G
SEQ ID NO: 6—HCDR1 of C1.2
SEQ ID NO: 7—HCDR2 of C1.2
SEQ ID NO: 8—HCDR3 of C1.2
SEQ ID NO: 9—LCDR1 of C1.2
SEQ ID NO: 10—LCDR2 of C1.2
SEQ ID NO: 11—LCDR3 of C1.2
SEQ ID NO: 12—consensus sequence of HCDR3 of C1.2
SEQ ID NO: 13—consensus sequence of LCDR3 of C1.2
SEQ ID NO: 14—Heavy chain of C1.2G with stabilized IgG4 constant region
SEQ ID NO: 15—Light chain of C1.2G with kappa constant region
SEQ ID NO: 16—sequence of exemplary h-G-CSFR
SEQ ID NO: 17—polypeptide comprising Ig and CRH domains of Macaca fascicularis G-CSFR (cynoG-CSFR) with a C-terminal polyhistidine tag
SEQ ID NO: 18—Heavy chain of C1.2G with stabilized IgG4 constant region and lacking C-terminal lysine.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only.

Functionally-equivalent products, compositions and methods are clearly within the scope of the present disclosure.

Any example of the present disclosure herein shall be taken to apply mutatis mutandis to any other example of the disclosure unless specifically stated otherwise. Stated another way, any specific example of the present disclosure may be combined with any other specific example of the disclosure (except where mutually exclusive).

Any example of the present disclosure disclosing a specific feature or group of features or method or method steps will be taken to provide explicit support for disclaiming the specific feature or group of features or method or method steps.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, antibodies and fragments thereof herein may be further clarified by the discussion in Kabat Sequences of Proteins of Immunological Interest, National Institutes of Health, Bethesda, Md., 1987 and 1991.

The term “EU numbering system of Kabat” will be understood to mean the numbering of an antibody heavy chain is according to the EU index as taught in Kabat et al., 1991, Sequences of Proteins of Immunological Interest, 5th Ed., United States Public Health Service, National Institutes of Health, Bethesda. The EU index is based on the residue numbering of the human IgG1 EU antibody.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

Reference herein to “granulocyte colony-stimulating factor” (G-CSF) includes native forms of G-CSF, mutant forms thereof, e.g., filgrastim and pegylated forms of G-CSF or filgrastim. This term also encompasses mutant forms of G-CSF retaining activity to bind to G-CSFR (e.g., human G-CSFR) and induce signaling.

G-CSF is a major regulator of granulocyte production. G-CSF is produced by bone marrow stromal cells, endothelial cells, macrophages, and fibroblasts, and production is induced by inflammatory stimuli. G-CSF acts through the G-CSF receptor (G-CSFR), which is expressed on early myeloid progenitors, mature neutrophils, monocytes/macrophages, T and B lymphocytes and endothelial cells.

For the purposes of nomenclature only and not limitation, an exemplary sequence of a human G-CSFR is set out in NCBI Reference Sequence: NP 000751.1 (and set out in SEQ ID NO: 16). The sequence of G-CSFR from other species can be determined using sequences provided herein and/or in publically available databases and/or determined using standard techniques (e.g., as described in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989)) Reference to human G-CSFR may be abbreviated to hG-CSFR and reference to cynomolgus monkey G-CSFR may be abbreviated to cynoG-CSFR. Reference to soluble G-CSFR refers to polypeptides comprising the ligand binding region of G-CSFR. The Ig and CRH domains of the G-CSFR are involved in ligand binding and receptor dimerization (Layton et al., J. Biol Chem., 272: 29735-29741, 1997 and Fukunaga et al, EMBO J. 10: 2855-2865, 1991). Soluble forms of G-CSFR comprising these portions of the receptor have been used in various studies of the receptor and mutation of the free cysteines at positions 78, 163, and 228 of the receptor assists in expression and isolation of the soluble receptor polypeptide (Mine et al., Biochem., 43: 2458-2464 2004) without affecting ligand binding.

The term “organic acid buffer” refers to conventional buffers of organic acids and salts. Suitable organic acid buffers for use in the formulation of the present disclosure are described herein.

The term “non-ionic surfactant” as used herein refers to any detergent that has an uncharged polar head. Suitable surfactants for use in the formulation of the present disclosure are described herein.

A “stable” formulation is one in which the protein in the formulation essentially retains its physical stability and/or chemical stability and/or biological activity upon storage.

In the context of the present disclosure, the term “monomer” or “monomeric” refers to the correctly folded protein (e.g., antibody or antigen binding fragment thereof). For example, a monomer of an antibody according to the present disclosure relates to the standard tetrameric antibody comprising two identical, glycosylated heavy and light chains respectively. An “aggregate” is a non-specific association of two or more protein molecules (e.g., high molecular weight species).

As used herein, the term “amino acid stabiliser” refers to an amino acid or derivative thereof that improves or otherwise enhances the stability of the formulation.

As used herein, the term “polyol” refers to a substance having a plurality of hydroxyl groups.

The term “dynamic viscosity” or “absolute viscosity” refers to the internal resistance to flow exhibited by a fluid at a specified temperature (e.g., 20° C.), the ratio of shearing stress to rate of shear. A liquid has a dynamic viscosity of one poise if a force of 1 dyne/square centimetre causes two parallel liquid surfaces one square centimetre in area and one square centimetre apart to move past one another at a velocity of 1 cm/second. One poise equals one hundred centipoise (cP) and one centipoise equals one millipascal-second (mPa*s) in System International (SI) units.

As used herein, the term “osmolality” is a measure of the osmoles (Osm) of solute per kilogram of solvent (osmol/kg or Osm/kg).

As used herein, the term “binds” is a reference to an interaction of a protein with another molecule that is dependent upon the presence of a particular structure (e.g., an antigenic determinant or epitope) on that molecule. For example, an antibody, or antigen binding fragment thereof, recognises and binds to a specific protein structure rather than to proteins generally. If an antibody binds to epitope “A”, the presence of a molecule containing epitope “A” (or free, unlabelled “A”), in a reaction containing labelled “A” and the protein, will reduce the amount of labelled “A” bound to the antibody.

As used herein, the term “specifically binds” or “binds specifically” shall be taken to mean that a protein described herein reacts or associates more frequently, more rapidly, with greater duration and/or with greater affinity with a particular molecule (e.g., antigen) than it does with alternative molecules. For example, a protein may bind to G-CSFR (e.g., hG-CSFR) with materially greater affinity (e.g., 20 fold or 40 fold or 60 fold or 80 fold to 100 fold or 150 fold or 200 fold) than it does to other cytokine receptor or to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

For the purposes of clarification and as will be apparent to the skilled artisan based on the exemplified subject matter herein, reference to “affinity” in this specification is a reference to K_{D} of a protein or antibody. For the purposes of clarification and as will be apparent to the skilled artisan based on the description herein, reference to an “affinity of at least about” will be understood to mean that the affinity (or K_{D}) is equal to the recited value or higher (i.e., the value recited as the affinity is lower), i.e., an affinity of 2 nM is greater than an affinity of 3 nM. Stated another way, this term could be “an affinity of X or less”, wherein X is a value recited herein.

The term “recombinant” shall be understood to mean the product of artificial genetic recombination. Accordingly, in the context of a protein comprising an antigen binding domain described herein, this term does not encompass an antibody naturally occurring within a subject's body that is the product of natural recombination that occurs during B cell maturation. However, if such an antibody is isolated, it is to be considered an isolated protein comprising an antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “antigen binding domain” or “antigen binding site” shall be taken to mean a structure formed by a protein that is capable of binding or specifically binding to an antigen. The antigen binding domain need not be a series of contiguous amino acids, or even amino acids in a single polypeptide chain. For example, in a Fv produced from two different polypeptide chains the antigen binding domain is made up of a series of amino acids of a VL and a V_{H} that interact with the antigen and that are generally, however not always in the one or more of the CDRs in each variable region. In some examples, an antigen binding domain is or comprises a V_{H} or a V_{L} or a Fv. In some examples, the antigen binding domain comprises one or more CDRs of an antibody.

The skilled artisan will be aware that an “antibody” is generally considered to be a protein that comprises a variable region made up of a plurality of polypeptide chains, e.g., a polypeptide comprising a V_{L} and a polypeptide comprising a V_{H}. An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies and chimeric antibodies.

The terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable region the presence of which are necessary for antigen binding. Each variable region typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol. Biol. 196: 901-917, 1987; Chothia et al. Nature 342, 877-883, 1989; and/or Al-Lazikani et al., J. Mol. Biol 273: 927-948, 1997; the IMGT numbering system of Lefranc et al., Devel. And Compar. Immunol., 27: 55-77, 2003; or the AHO numbering system of Honnegher and Plükthun J. Mol. Biol., 309: 657-670, 2001. For example, according to the numbering system of Kabat, V_{H} framework regions (FRs) and CDRs are positioned as follows: residues 1-30 (FR1), 31-(CDR1), 36-49 (FR2), 50-65 (CDR2), 66-94 (FR3), 95-102 (CDR3) and 103-113 (FR4). According to the numbering system of Kabat, VL FRs and CDRs are positioned as follows: residues 1-23 (FR1), 24-34 (CDR1), 35-49 (FR2), 50-56 (CDR2), 57-88 (FR3), 89-97 (CDR3) and 98-107 (FR4). The present disclosure is not limited to FRs and CDRs as defined by the Kabat numbering system, but includes all numbering systems, including those discussed above. In one example, reference herein to a CDR (or a FR) is in respect of those regions according to the Kabat numbering system.

“Framework regions” (FRs) are those variable region residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the VL is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

The term “fragment crystallisable” or “Fc” or “Fc region” or “Fc portion” (which can be used interchangeably herein) refers to a region of an antibody comprising at least one constant domain and which is generally (though not necessarily) glycosylated and which is capable of binding to one or more Fc receptors and/or components of the complement cascade. The heavy chain constant region can be selected from any of the five isotypes: α, δ, ε, γ, or μ. Furthermore, heavy chains of various subclasses (such as the IgG subclasses of heavy chains) are responsible for different effector functions and thus, by choosing the desired heavy chain constant region, proteins with desired effector function can be produced. Exemplary heavy chain constant regions are gamma 1 (IgG₁), gamma 2 (IgG₂), gamma 3 (IgG₃) and gamma 4 (IgG₄), or hybrids thereof.

The term “constant region” as used herein, refers to a portion of heavy chain or light chain of an antibody other than the variable region. In a heavy chain, the constant region generally comprises a plurality of constant domains and a hinge region, e.g., a IgG constant region comprises the following linked components, a constant heavy C_{H} C_{H}1, a linker, a C_{H}2 and a C_{H}3. In a light chain, a constant region generally comprises one constant domain (a C_{L}1).

The term “stabilised IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognising two distinct antigens (resulting in bispecific molecules). Fab arm exchange occurs naturally in vivo and can be induced in vitro by purified blood cells or reducing agents such as reduced glutathione.

As used herein, the term “monospecific” refers to a binding domain comprising one or more antigen binding sites each with the same epitope specificity. Thus, a monospecific binding domain can comprise a single antigen binding site (e.g., a Fv, scFv, Fab, etc) or can comprise several antigen binding sites that recognise the same epitope (e.g., are identical to one another), e.g., a diabody or an antibody. The requirement that the binding region is “monospecific” does not mean that it binds to only one antigen, since multiple antigens can have shared or highly similar epitopes that can be bound by a single antigen binding site. A monospecific binding domain that binds to only one antigen is said to “exclusively bind” to that antigen.

The term “multispecific” refers to a binding domain comprising two or more antigen binding sites, each of which binds to a distinct epitope, for example each of which binds to a distinct antigen. For example, the multispecific binding domain may include antigen binding sites that recognise two or more different epitopes of the same protein (e.g., coagulation factor) or that may recognise two or more different epitopes of different proteins (i.e., distinct coagulation factors). In one example, the binding domain may be “bispecific”, that is, it includes two antigen binding sites that specifically bind two distinct epitopes. For example, a bispecific binding domain specifically binds or has specificities for two different epitopes on the same protein. In another example, a bispecific binding domain specifically binds two distinct epitopes on two different proteins.

The term “competitively inhibits” shall be understood to mean that a protein of the disclosure (or an antigen binding site thereof) reduces or prevents binding of a recited antibody or protein to G-CSF or G-CSFR, e.g., to hG-CSFR. This may be due to the protein (or antigen binding site) and antibody binding to the same or an overlapping epitope. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Preferably, the protein reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to G-CSF or G-CSFR either in the presence or absence of the protein. If less antibody binds in the presence of the protein than in the absence of the protein, the protein is considered to competitively inhibit binding of the antibody. In one example, the competitive inhibition is not due to steric hindrance.

As used herein, the term “epitope” (syn. “antigenic determinant”) shall be understood to mean a region of hG-CSFR to which a protein comprising an antigen binding site of an antibody binds. This term is not necessarily limited to the specific residues or structure to which the protein makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when hG-CSFR is folded, i.e., a “conformational epitope”. For example, a conformational epitope in hG-CSFR comprises amino acids in one or more or two or more or all of the regions corresponding to 111-115, 170-176, 218-234 and/or 286-300 of SEQ ID NO: 1. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

“Overlapping” in the context of two epitopes shall be taken to mean that two epitopes share a sufficient number of amino acid residues to permit a protein (or antigen binding site thereof) that binds to one epitope to competitively inhibit the binding of a protein (or antigen binding site) that binds to the other epitope. For example, the “overlapping” epitopes share at least 1 or 2 or 3 or 4 or 5 or 6 or 7 or 8 or 9 or 20 amino acids.

The phrase “conservative amino acid substitution” refers to replacement or substitution of an amino acid residue with an amino acid residue having a similar side chain and/or hydropathicity and/or hydrophilicity. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Hydropathic indices are described, for example in Kyte and Doolittle J. Mol. Biol., 157: 105-132, 1982 and hydrophilic indices are described in, e.g., U.S. Pat. No. 4,554,101.

As used herein, the terms “disease”, “disorder” or “condition” refers to a disruption of or interference with normal function, and is not to be limited to any specific condition, and will include diseases or disorders.

As used herein, the terms “treating”, “treat” or “treatment” include administering a protein described herein to thereby reduce or eliminate at least one symptom of a specified disease or condition or to slow progression of the disease or condition.

As used herein, the terms “preventing”, “prevent” or “prevention” includes providing prophylaxis with respect to occurrence or recurrence of a specified disease or condition in an individual. An individual may be predisposed to or at risk of developing the disease or disease relapse but has not yet been diagnosed with the disease or the relapse.

As used herein, a subject “at risk” of developing a disease or condition or relapse thereof or relapsing may or may not have detectable disease or symptoms of disease, and may or may not have displayed detectable disease or symptoms of disease prior to the treatment according to the present disclosure. “At risk” denotes that a subject has one or more risk factors, which are measurable parameters that correlate with development of the disease or condition, as known in the art and/or described herein.

As used herein, the term “subject” shall be taken to mean any animal including humans, for example a mammal. Exemplary subjects include but are not limited to humans and non-human primates. For example, the subject is a human.

### Proteins of the Pharmaceutical Formulation

As discussed herein, the present disclosure provides a liquid pharmaceutical formulation comprising a protein comprising an antigen binding domain that binds to or specifically binds to G-CSFR. In some examples, the protein comprises at least a V_{H} and a V_{L}, wherein the V_{H} and V_{L} bind to form a Fv comprising an antigen binding domain.

### Proteins Comprising Antigen Binding Domains

In one example, the protein comprising an antigen binding domain that binds to or specifically binds to G-CSFR is an antibody or antigen binding fragment. For example, the protein is an antibody or antigen binding fragment that binds to G-C SFR.

Methods for generating antibodies are known in the art and/or described in Harlow and Lane (editors) Antibodies: A Laboratory Manual, Cold Spring Harbor Laboratory, (1988). Generally, in such methods G-CSFR or a region thereof (e.g., an extracellular domain) or immunogenic fragment or epitope thereof or a cell expressing and displaying same (i.e., an immunogen), optionally formulated with any suitable or desired carrier, adjuvant, or pharmaceutically acceptable excipient, is administered to a non-human animal, for example, a mouse, chicken, rat, rabbit, guinea pig, dog, horse, cow, goat or pig. The immunogen may be administered intranasally, intramuscularly, subcutaneously, intravenously, intradermally, intraperitoneally, or by other known route.

Monoclonal antibodies are one exemplary form of an antibody contemplated by the present disclosure. The term “monoclonal antibody” or “mAb” refers to a homogeneous antibody population capable of binding to the same antigen(s), for example, to the same epitope within the antigen. This term is not intended to be limited as regards to the source of the antibody or the manner in which it is made.

For the production of mAbs any one of a number of known techniques may be used, such as, for example, the procedure exemplified in U.S. Pat. No. 4,196,265 or Harlow and Lane (1988), supra.

Alternatively, ABL-MYC technology (NeoClone, Madison WI 53713, USA) is used to produce cell lines secreting MAbs (e.g., as described in Largaespada et al, J. Immunol. Methods. 197: 85-95, 1996).

Antibodies can also be produced or isolated by screening a display library, e.g., a phage display library, e.g., as described in U.S. Pat. No. 6,300,064 and/or U.S. Pat. No. 5,885,793. For example, the present inventors have isolated fully human antibodies from a phage display library.

The antibody of the present disclosure may be a synthetic antibody. For example, the antibody is a chimeric antibody, a humanised antibody, a human antibody or a de-immunised antibody.

The antibodies or antigen binding fragments of the present disclosure may be humanised.

The term “humanised antibody” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanised antibodies also include antibodies in which one or more residues of the human protein are modified by one or more amino acid substitutions and/or one or more FR residues of the human antibody are replaced by corresponding non-human residues. Humanised antibodies may also comprise residues which are found in neither the human antibody or in the non-human antibody. Any additional regions of the antibody (e.g., Fc region) are generally human. Humanisation can be performed using a method known in the art, e.g., U.S. Pat. Nos. 5,225,539, 6,054,297, 7,566,771 or U.S. Pat. No. 5,585,089. The term “humanised antibody” also encompasses a super-humanised antibody, e.g., as described in U.S. Pat. No. 7,732,578. A similar meaning will be taken to apply to the term “humanised antigen binding fragment”.

The antibodies or antigen binding fragments thereof of the present disclosure may be human antibodies or antigen binding fragments thereof. The term “human antibody” as used herein refers to antibodies having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human antibody will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516. A similar meaning will be taken to apply to the term “human antigen binding fragment”.

The antibodies or antigen binding fragments thereof of the present disclosure may be synhumanised antibodies or antigen binding fragments thereof. The term “synhumanised antibody” refers to an antibody prepared by a method described in WO2007019620. A synhumanised antibody includes a variable region of an antibody, wherein the variable region comprises FRs from a New World primate antibody variable region and CDRs from a non-New World primate antibody variable region.

The antibody or antigen binding fragment thereof of the present disclosure may be primatised. A “primatised antibody” comprises variable region(s) from an antibody generated following immunisation of a non-human primate (e.g., a cynomolgus macaque). Optionally, the variable regions of the non-human primate antibody are linked to human constant regions to produce a primatised antibody. Exemplary methods for producing primatised antibodies are described in U.S. Pat. No. 6,113,898.

In one example an antibody or antigen binding fragment thereof of the disclosure is a chimeric antibody or fragment. The term “chimeric antibody” or “chimeric antigen binding fragment” refers to an antibody or fragment in which one or more of the variable domains is from a particular species (e.g., murine, such as mouse or rat) or belonging to a particular antibody class or subclass, while the remainder of the antibody or fragment is from another species (such as, for example, human or non-human primate) or belonging to another antibody class or subclass. In one example, a chimeric antibody comprising a V_{H} and/or a V_{L} from a non-human antibody (e.g., a murine antibody) and the remaining regions of the antibody are from a human antibody. The production of such chimeric antibodies and antigen binding fragments thereof is known in the art, and may be achieved by standard means (as described, e.g., in U.S. Pat. Nos. 6,331,415; 5,807,715; 4,816,567 and 4,816,397).

The present disclosure also contemplates a deimmunised antibody or antigen binding fragment thereof, e.g., as described in WO2000034317 and WO2004108158. De-immunised antibodies and fragments have one or more epitopes, e.g., B cell epitopes or T cell epitopes removed (i.e., mutated) to thereby reduce the likelihood that a subject will raise an immune response against the antibody or protein. For example, an antibody of the disclosure is analysed to identify one or more B or T cell epitopes and one or more amino acid residues within the epitope is mutated to thereby reduce the immunogenicity of the antibody.

Exemplary human antibodies are described herein and include C1.2 and C1.2G and/or variable regions thereof. These human antibodies provide an advantage of reduced immunogenicity in a human compared to non-human antibodies. Exemplary antibodies are described in WO2012/171057.

### Bispecific Antibodies

In one example, the protein of the present disclosure may be a bispecific antibody or fragment thereof. For example, the antibody or fragment may bind to G-CSFR, and another target. A bispecific antibody is a molecule comprising two types of antibodies or antibody fragments (e.g., two half antibodies) having specificities for different antigens or epitopes. Exemplary bispecific antibodies bind to two different epitopes of the same protein. Alternatively, the bispecific antibody binds to two different epitopes on two different proteins.

Exemplary “key and hole” or “knob and hole” bispecific proteins as described in U.S. Pat. No. 5,731,168. In one example, a constant region (e.g., an IgG₄ constant region) comprises a T366W mutation (or knob) and a constant region (e.g., an IgG₄ constant region) comprises a T366S, L368A and Y407V mutation (or hole). In another example, the first constant region comprises T350V, T366L, K392L and T394W mutations (knob) and the second constant region comprises T350V, L351Y, F405A and Y407V mutations (hole).

Methods for generating bispecific antibodies are known in the art and exemplary methods are described herein.

In one example, an IgG type bispecific antibody is secreted by a hybrid hybridoma (quadroma) formed by fusing two types of hybridomas that produce IgG antibodies (Milstein C et al., Nature 1983, 305: 537-540). In another example, the antibody can be secreted by introducing into cells genes of the L chains and H chains that constitute the two IgGs of interest for co-expression (Ridgway, J B et al. Protein Engineering 1996, 9: 617-621; Merchant, A M et al. Nature Biotechnology 1998, 16: 677-681).

In one example, a bispecific antibody fragment is prepared by chemically cross-linking Fab's derived from different antibodies (Keler T et al. Cancer Research 1997, 57: 4008-4014).

In one example, a leucine zipper derived from Fos and Jun or the like is used to form a bispecific antibody fragment (Kostelny S A et al. J. of Immunology, 1992, 148: 1547-53).

In one example, a bispecific antibody fragment is prepared in a form of diabody comprising two crossover scFv fragments (Holliger P et al. Proc. of the National Academy of Sciences of the USA 1993, 90: 6444-6448).

### Antibody Fragments

As described herein, a protein of the disclosure comprises an antigen binding fragment of an antibody. Exemplary antigen binding fragments for use in the present disclosure are described below.

### Single-Domain Antibodies

In some examples, an antigen binding fragment of an antibody of the disclosure is or comprises a single-domain antibody (which is used interchangeably with the term “domain antibody” or “dAb”). A single-domain antibody is a single polypeptide chain comprising all or a portion of the heavy chain variable domain of an antibody.

### Diabodies, Triabodies, Tetrabodies

In some examples, an antigen binding fragment of the disclosure is or comprises a diabody, triabody, tetrabody or higher order protein complex such as those described in WO98/044001 and/or WO94/007921.

For example, a diabody is a protein comprising two associated polypeptide chains, each polypeptide chain comprising the structure V_{L}-X-V_{H} or V_{H}-X-V_{L}, wherein X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form a Fv molecule capable of specifically binding to one or more antigens. The VL and V_{H} can be the same in each polypeptide chain or the V_{L} and V_{H} can be different in each polypeptide chain so as to form a bispecific diabody (i.e., comprising two Fvs having different specificity).

Single Chain Fv (scFv) Fragments

The skilled artisan will be aware that scFvs comprise V_{H} and V_{L} regions in a single polypeptide chain and a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favoured linkers for a scFv.

In one example, the linker comprises the sequence SGGGGSGGGGSGGGGS.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv.

Alternatively, or in addition, the present disclosure encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

In some examples, an antigen binding fragment of the disclosure is or comprises a heavy chain antibody. Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR). A general description of heavy chain antibodies from camelids and the variable regions thereof and methods for their production and/or isolation and/or use is found inter alia in the following references WO 94/04678, WO 97/49805 and WO 97/49805. A general description of heavy chain antibodies from cartilaginous fish and the variable regions thereof and methods for their production and/or isolation and/or use is found inter alia in WO 2005/118629.

### Half-Antibodies

In some examples, the antigen binding fragment of the present disclosure is a half-antibody or a half-molecule. The skilled artisan will be aware that a half antibody refers to a protein comprising a single heavy chain and a single light chain. The term “half antibody” also encompasses a protein comprising an antibody light chain and an antibody heavy chain, wherein the antibody heavy chain has been mutated to prevent association with another antibody heavy chain. In one example, a half antibody forms when an antibody dissociates to form two molecules each containing a single heavy chain and a single light chain.

Methods for generating half antibodies are known in the art and exemplary methods are described herein.

In one example, the half antibody can be secreted by introducing into cells genes of the single heavy chain and single light chain that constitute the IgG of interest for expression. In one example, a constant region (e.g., an IgG4 constant region) comprises a “key or hole” (or “knob or hole”) mutation to prevent heterodimer formation. In one example, a constant region (e.g., an IgG4 constant region) comprises a T366W mutation (or knob). In another example, a constant region (e.g., an IgG4 constant region) comprises a T366S, L368A and Y407V mutation (or hole). In another example, the constant region comprises T350V, T366L, K392L and T394W mutations (knob). In another example, the constant region comprises T350V, L351Y, F405A and Y407V mutations (hole). Exemplary constant region amino acid substitutions are numbered according to the EU numbering system.

### Other Antibodies and Antibody Fragments

The present disclosure also contemplates other antibodies and antibody fragments, such as:
(i) minibodies, e.g., as described in U.S. Pat. No. 5,837,821;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fab₃ (e.g., as described in EP19930302894).

### Stabilised Proteins

Proteins of the present disclosure can comprise an IgG₄ constant region or a stabilized IgG₄ constant region. The term “stabilised IgG₄ constant region” will be understood to mean an IgG₄ constant region that has been modified to reduce Fab arm exchange or the propensity to undergo Fab arm exchange or formation of a half-antibody or a propensity to form a half antibody. “Fab arm exchange” refers to a type of protein modification for human IgG₄, in which an IgG₄ heavy chain and attached light chain (half-molecule) is swapped for a heavy-light chain pair from another IgG₄ molecule. Thus, IgG₄ molecules may acquire two distinct Fab arms recognizing two distinct antigens (resulting in bispecific molecules). Fab arm exchange occurs naturally in vivo and can be induced in vitro by purified blood cells or reducing agents such as reduced glutathione.

In one example, a stabilised IgG₄ constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG₄, this residue is generally a serine. Following substitution of the serine for proline, the IgG₄ hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG₁ according to the numbering system of Kabat. Hinge regions of other IgG isotypes may be aligned with the IgG₁ sequence by placing the first and last cysteine residues forming inter-heavy chain disulphide (S—S) bonds in the same positions (see for example WO2010080538).

### Preparation of the Pharmaceutical Formulation

As described herein, the formulations of the present disclosure comprise an organic acid buffer, a non-ionic surfactant and at least one amino acid stabiliser. In some examples, the formulation has a pH of 5.0 to 6.0. Preparation of the pharmaceutical formulation is performed according to standard methods known in the art and/or according to methods described herein.

### Organic Acid Buffers

The skilled person will understand that organic acid buffers suitable for use in the present disclosure comprise one or more carboxylic acid or acid phenolic groups without basic amino groups. In addition to the buffering capacity provided by the acidic groups, such organic buffers used herein can contain additional ionisable functionality provided by, for example, an amino group.

It will be apparent to the skilled person that buffers suitable for use in the present disclosure will be stable and effective at the desired pH and will provide sufficient buffer capacity to maintain the desired pH over the range of conditions to which it will be exposed during formulation and storage of the product. For example, a stable buffer will provide thermal aggregation stability (e.g., during freeze/thaw or elevated temperatures), not be affected by oxidation of physical degradation (e.g., insoluble particulate formation) and provide the desired polydispersity (i.e., particle distribution). Suitable buffers will not form deleterious complexes with metal ions, be toxic, or unduly penetrate, solubilise, or absorb on membranes or other surfaces. Furthermore, the skilled person will recognise that such buffers should not interact with other components of the composition in any manner which decreases their availability or effectiveness. Additionally, the buffering agent of the pharmaceutical formulation must be safe for administration, compatible with other components of the composition over the shelf-life of the product, and acceptable for administration to the subject.

Suitable organic acid buffers for use in the present disclosure will be apparent to the skilled person and include, for example, histidine buffers (e.g., histidine chloride, histidine acetate, histidine phosphate, histidine sulfate, etc.), glutamate buffers (e.g., monosodium glutamate, etc.), citrate buffers (e.g. monosodium citrate-disodium citrate mixture, citric acid-trisodium citrate mixture, citric acid-monosodium citrate mixture, etc.), succinate buffers (e.g. succinic acid-monosodium succinate mixture, succinic acid-sodium hydroxide mixture, succinic acid-disodium succinate mixture, etc.), tartrate buffers (e.g. tartaric acid-sodium tartrate mixture, tartaric acid-potassium tartrate mixture, tartaric acid-sodium hydroxide mixture, etc.), fumarate buffers (e.g. fumaric acid-monosodium fumarate mixture, fumaric acid-disodium fumarate mixture, monosodium fumarate-disodium fumarate mixture, etc.) gluconate buffers (e.g. gluconic acid-sodium gluconate mixture, gluconic acid-sodium hydroxide mixture, gluconic acid-potassium gluconate mixture, etc.), oxalate buffers (e.g. oxalic acid-sodium oxalate mixture, oxalic acid-sodium hydroxide mixture, oxalic acid-potassium oxalate mixture, etc.), lactate buffers (e.g. lactic acid-sodium lactate mixture, lactic acid-sodium hydroxide mixture, lactic acid-potassium lactate mixture, etc.) and acetate buffers (e.g. acetic acid-sodium acetate mixture, acetic acid-sodium hydroxide mixture, etc.).

In one example of the present disclosure, the organic acid buffer is selected from the group consisting of a histidine buffer, a glutamate buffer, a succinate buffer and a citrate buffer. For example, the organic acid buffer is a histidine buffer. For example, the organic acid buffer is L-histidine.

Methods of assessing the suitability of buffers will be apparent to the skilled person and/or described herein and include, for example, differential scanning fluorimetry and dynamic light scattering.

### Non-Ionic Surfactants

The amount of non-ionic surfactant added to the pharmaceutical formulation will be apparent to the skilled person and is in an amount such that it suppresses aggregation (e.g., by preventing surface denaturation), increases stabilisation (e.g., during thermal and/or physical stress), minimises the formation of particulates in the formulation (e.g., sub-visible and/or visible particle formation), reduces surface adsorption and/or assists in protein refolding.

Suitable non-ionic surfactants for use in the present disclosure will be apparent to the skilled person and include, for example, polyoxyethylensorbitan fatty acid esters (e.g., polysorbate 20 and polysorbate 80), polyethylene-polypropylene copolymers, polyethylene-polypropylene glycols, polyoxyethylene-stearates, polyoxyethylene alkyl ethers, e.g. polyoxyethylene monolauryl ether, alkylphenylpolyoxyethylene ethers (Triton-X), polyoxyethylene-polyoxypropylene copolymer (Poloxamer, Pluronic), sodium dodecyl sulphate (SDS).

In one example of the present disclosure, the non-ionic surfactant is selected from the group consisting of polyoxyethylensorbitan fatty acid esters and polyoxyethylene-polyoxypropylene copolymers. For example, the polyoxyethylensorbitan fatty acid ester is polyoxyethylene sorbitan monooleate (i.e., polysorbate 80) or polyoxyethylene sorbitan monolaurate (polysorbate 20).

### Amino Acid Stabilisers

The amount of amino acid stabiliser(s) added to the pharmaceutical formulation will be apparent to the skilled person and is in an amount that such that it reduces thermal and/or physical stress (e.g., freeze/thaw or agitation), and/or confers or enhances stability of the protein.

Suitable amino acids for use in the present disclosure will be apparent to the skilled person and include, for example, glycine, alanine, valine, leucine, isoleucine, methionine, threonine, phenylalanine, tyrosine, serine, cysteine, histidine, tryptophan, proline, aspartic acid, glutamic acid, arginine, lysine, ornithine and asparagine and salts thereof.

In one example of the present disclosure, the at least one amino acid is selected from the group consisting of proline, arginine and methionine. For example, the at least one amino acid stabiliser includes proline or a salt form thereof. For example, the at least one amino acid stabiliser includes arginine or a salt form thereof. For example, the amino acid stabilisers are proline and arginine or a salt form thereof.

### Protein Production

Methods of producing and obtaining proteins for use in the formulation described herein will be known to those skilled in the art. For example, in the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing an protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant proteins are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding an protein (e.g., derived from the information provided herein), an enhancer element, a promoter, and a transcription termination sequence. The skilled artisan will be aware of suitable sequences for expression of an protein. Exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CVI line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHOS promoter, the nmt promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Isolation of Proteins

Where a protein (e.g., antibody) is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

### Assaying the Pharmaceutical Formulation and Proteins of the Disclosure

High concentration pharmaceutical formulations of the present disclosure are readily screened for physical and biological activity and/or stability using methods known in the art and/or as described below.

### Binding to G-CSFR and Mutants Thereof

It will be apparent to the skilled artisan from the disclosure herein that some proteins described herein bind to the ligand binding domain of hG-CSFR and to specific mutant forms of the ligand binding domain of hG-CSFR (e.g., SEQ ID NO: 1 without or with certain point mutations) and/or bind to both human and cynomolgus monkey G-C SFR. Methods for assessing binding to a target are known in the art, e.g., as described in Scopes (In: Protein purification: principles and practice, Third Edition, Springer Verlag, 1994). Such a method generally involves labeling the target and contacting it with immobilized protein. Following washing to remove non-specific bound target, the amount of label and, as a consequence, bound target is detected. Of course, the target can be immobilized and the protein can be labeled. Panning-type assays can also be used. Alternatively, or additionally, surface plasmon resonance assays can be used.

The assays described above can also be used to detect the level of binding of a protein to hG-C SFR or a ligand binding domain thereof (e.g., SEQ ID NO: 1) or mutant form thereof.

In one example, a protein of the present disclosure binds to a polypeptide of SEQ ID NO: 1 in which an alanine is substituted for the lysine at position 167 of SEQ ID NO: 1 and/or in which an alanine is substituted for the histidine at position 168 of SEQ ID NO: 1 at substantially the same level (e.g., within 10% or 5% or 1%) as it binds to SEQ ID NO: 1.

In one example, a protein of the present disclosure binds to a polypeptide of SEQ ID NO: 1 in which an alanine is substituted for the arginine at position 287 of SEQ ID NO: 1 at a level at least about 100 fold or 150 fold or 160 fold or 200 fold lower than it binds to a polypeptide of SEQ ID NO: 1. In one example, a protein of the present disclosure binds to a polypeptide of SEQ ID NO: 1 in which an alanine is substituted for the arginine at position 287 of SEQ ID NO: 1 at a level at least about 160 fold lower than it binds to a polypeptide of SEQ ID NO: 1.

In one example, a protein of the present disclosure binds to a polypeptide of SEQ ID NO: 1 in which an alanine is substituted for the histidine at position 237 of SEQ ID NO: 1 at a level at least about 20 fold or 40 fold or 50 fold or 60 fold lower than it binds to a polypeptide of SEQ ID NO: 1. In one example, a protein of the present disclosure binds to a polypeptide of SEQ ID NO: 1 in which an alanine is substituted for the histidine at position 237 of SEQ ID NO: 1 at a level at least about 50 fold lower than it binds to a polypeptide of SEQ ID NO: 1.

In one example, a protein of the present disclosure binds to a polypeptide of SEQ ID NO: 1 in which an alanine is substituted for the methionine at position 198 of SEQ ID NO: 1 at a level at least about 20 fold or 40 fold or 60 fold or 70 fold lower than it binds to a polypeptide of SEQ ID NO: 1. In one example, a protein of the present disclosure binds to a polypeptide of SEQ ID NO: 1 in which an alanine is substituted for the methionine at position 198 of SEQ ID NO: 1 at a level at least about fold lower than it binds to a polypeptide of SEQ ID NO: 1.

In one example, a protein of the present disclosure binds to a polypeptide of SEQ ID NO: 1 in which an alanine is substituted for the tyrosine at position 172 of SEQ ID NO: 1 at a level at least about 20 fold or 30 fold or 40 fold lower than it binds to a polypeptide of SEQ ID NO: 1. In one example, a protein of the present disclosure binds to a polypeptide of SEQ ID NO: 1 in which an alanine is substituted for the tyrosine at position 172 of SEQ ID NO: 1 at a level at least about 40 fold lower than it binds to a polypeptide of SEQ ID NO: 1.

In one example, a protein of the present disclosure binds to a polypeptide of SEQ ID NO: 1 in which an alanine is substituted for the leucine at position 171 of SEQ ID NO: 1 at a level at least about 100 fold or 120 fold or 130 fold or 140 fold lower than it binds to a polypeptide of SEQ ID NO: 1. In one example, a protein of the present disclosure binds to a polypeptide of SEQ ID NO: 1 in which an alanine is substituted for the leucine at position 171 of SEQ ID NO: 1 at a level at least about 140 fold lower than it binds to a polypeptide of SEQ ID NO: 1.

In one example, a protein of the present disclosure binds to a polypeptide of SEQ ID NO: 1 in which an alanine is substituted for the leucine at a position 111 of SEQ ID NO: 1 at a level at least about 20 fold or 40 fold or 60 fold or 70 fold lower than it binds to a polypeptide of SEQ ID NO: 1. In one example, a protein of the present disclosure binds to a polypeptide of SEQ ID NO: 1 in which an alanine is substituted for the leucine at a position 111 of SEQ ID NO: 1 at a level at least about 60 fold lower than it binds to a polypeptide of SEQ ID NO: 1.

In one example, a protein of the present disclosure binds to a polypeptide of SEQ ID NO: 1 in which an alanine is substituted for the histidine at position 168 of SEQ ID NO: 1 at a level no more than 5 fold or 4 fold or 3 fold or 2 fold or 1 fold lower than it binds to a polypeptide of SEQ ID NO: 1.

In one example, a protein of the present disclosure binds to a polypeptide of SEQ ID NO: 1 in which an alanine is substituted for the lysine at position 167 of SEQ ID NO: 1 at a level no more than 5 fold or 4 fold or 3 fold or 2 fold or 1 fold lower than it binds to a polypeptide of SEQ ID NO: 1.

In some examples, the level of binding is conveniently determined using a biosensor.

The present disclosure contemplates any combination of the foregoing characteristics. In one example, a protein described herein has all of the binding characteristics set forth in the preceding seven paragraphs.

### Epitope Mapping

In another example, the epitope bound by a protein described herein is determined (i.e., mapped). Epitope mapping methods will be apparent to the skilled artisan. For example, a series of overlapping peptides spanning the hG-CSFR sequence or a region thereof comprising an epitope of interest, e.g., peptides comprising 10-15 amino acids are produced. The protein is then contacted to each peptide and the peptide(s) to which it binds determined. This permits determination of peptide(s) comprising the epitope to which the protein binds. If multiple non-contiguous peptides are bound by the protein, the protein may bind a conformational epitope.

Alternatively, or in addition, amino acid residues within hG-CSFR are mutated, e.g., by alanine scanning mutagenesis, and mutations that reduce or prevent protein binding are determined. Any mutation that reduces or prevents binding of the protein is likely to be within the epitope bound by the protein.

A further method is exemplified herein, and involves binding hG-CSFR or a region thereof to an immobilized protein of the present disclosure and digesting the resulting complex with proteases. Peptide that remains bound to the immobilized protein are then isolated and analyzed, e.g., using mass spectrometry, to determine their sequence.

A further method involves converting hydrogens in hG-CSFR or a region thereof to deutrons and binding the resulting protein to an immobilized protein of the present disclosure. The deutrons are then converted back to hydrogen, the hG-CSFR or region thereof isolated, digested with enzymes and analyzed, e.g., using mass spectrometry to identify those regions comprising deutrons, which would have been protected from conversion to hydrogen by the binding of a protein described herein.

Optionally, the dissociation constant (Kd) of a protein for hG-CSFR or an epitope thereof is determined. The “Kd” or “Kd value” for a hG-CSFR binding protein is in one example measured by a radiolabeled or fluorescently-labeled hG-CSFR binding assay. This assay equilibrates the protein with a minimal concentration of labeled G-CSFR in the presence of a titration series of unlabeled hG-CSFR. Following washing to remove unbound hG-CSFR, the amount of label is determined, which is indicative of the Kd of the protein.

According to another example the Kd or Kd value is measured by using surface plasmon resonance assays, e.g., using BIAcore surface plasmon resonance (BIAcore, Inc., Piscataway, NJ) with immobilized hG-CSFR or a region thereof.

In some examples, proteins having a similar Kd or a higher Kd than C1.2 or C1.2G are selected, because they are likely to compete for binding to hG-CSFR.

### Determining Competitive Binding

Assays for determining a protein that competitively inhibits binding of monoclonal antibody C1.2 or C1.2G will be apparent to the skilled artisan. For example, C1.2 or C1.2G is conjugated to a detectable label, e.g., a fluorescent label or a radioactive label. The labeled antibody and the test protein are then mixed and contacted with hG-CSFR or a region thereof (e.g., a polypeptide comprising SEQ ID NO: 1) or a cell expressing same. The level of labeled C1.2 or C1.2G is then determined and compared to the level determined when the labeled antibody is contacted with the hG-CSFR, region or cells in the absence of the protein. If the level of labeled C1.2 or C1.2G is reduced in the presence of the test protein compared to the absence of the protein, the protein is considered to competitively inhibit binding of C1.2 or C1.2G to hG-CSFR.

Optionally, the test protein is conjugated to different label to C1.2 or C1.2G. This alternate labeling permits detection of the level of binding of the test protein to hG-CSFR or the region thereof or the cell.

In another example, the protein is permitted to bind to hG-CSFR or a region thereof (e.g., a polypeptide comprising SEQ ID NO: 1) or a cell expressing same prior to contacting the hG-CSFR, region or cell with C1.2 or C1.2G. A reduction in the amount of bound C1.2 or C1.2G in the presence of the protein compared to in the absence of the protein indicates that the protein competitively inhibits C1.2 or C1.2G binding to hG-CSFR. A reciprocal assay can also be performed using labeled protein and first allowing C1.2 or C1.2G to bind to G-CSFR. In this case, a reduced amount of labeled protein bound to hG-CSFR in the presence of C1.2 or C1.2G compared to in the absence of C1.2 or C1.2G indicates that the protein competitively inhibits binding of C1.2 or C1.2G to hG-CSFR.

Any of the foregoing assays can be performed with a mutant form of hG-CSFR and/or SEQ ID NO: 1 and/or a ligand binding region of hG-CSFR to which C1.2 or C1.2G binds, e.g., as described herein.

### Determining Inhibition of G-CSF Signaling

In some examples of the present disclosure, a protein described herein is capable of inhibiting hG-CSFR signaling.

Various assays are known in the art for assessing the ability of a protein to inhibit signaling of a ligand through a receptor.

In one example, the protein reduces or prevents G-CSF binding to the hG-CSFR. These assays can be performed as a competitive binding assay as described herein using labeled G-CSF and/or labeled protein.

In another example, the protein reduces formation of CFU-G when CD34⁺ bone marrow cells are cultured in the presence of G-CSF. In such assays, CD34⁺ bone marrow cells are cultured in a semi-solid cell culture medium in the presence of G-CSF (e.g., about 10 ng/ml cell culture medium) and, optionally stem cell factor (e.g., about 10 ng/ml cell culture medium) in the presence or absence of a test protein. After a sufficient time for granulocyte clones (CFU-G) to form, the number of clones or colonies is determined. A reduction in the number of colonies in the presence of the protein compared to in the absence of the protein indicates that the protein inhibits G-CSF signaling. By testing multiple concentrations of the protein an IC₅₀ is determined, i.e., a concentration at which 50% of the maximum inhibition of CFU-G formation occurs. In one example, the IC₅₀ is 0.2 nM or less, such as 0.1 nM or less, for example, or less, or 0.08 nM or less, or 0.07 nM or less, or 0.06 nM or less or 0.05 nM or less. In one example, the IC₅₀ is 0.04 nM or less. In another example, the IC₅₀ is or less. The foregoing IC₅₀s relate to any CFU-G assay described herein.

In a further example, the protein reduces proliferation of cells (e.g., BaF3 cells) expressing hG-CSFR which are cultured in the presence of G-CSF. Cells are cultured in the presence of G-CSF (e.g., 0.5 ng/ml) and the presence or absence of a test protein. Methods for assessing cell proliferation are known in the art and include, for example, MTT reduction and thymidine incorporation. A protein that reduces the level of proliferation compared to the level observed in the absence of the protein is considered to inhibit G-CSF signaling. By testing multiple concentrations of the protein an IC₅₀ is determined, i.e., a concentration at which 50% of the maximum inhibition of cell proliferation occurs. In one example, the IC₅₀ is 6 nM or less, such as 5.9 nM or less. In another example, the IC₅₀ is 2 nM or less or 1 nM or less or 0.7 nM or cell or 0.6 nM or less or 0.5 nM or less. The foregoing IC₅₀s relate to any cell proliferation assay described herein.

In a further example, the protein reduces mobilization of hematopoietic stem cells and/or endothelial progenitor cells in vivo following G-CSF administration and/or reduces the number of neutrophils in vivo, e.g., following G-CSF administration (however this is not essential). For example, the protein is administered, optionally before, at the time of or after administration of G-CSF or a modified form thereof (e.g., PEGylated G-CSF or filgrastim). The number of hematopoietic stem cells (e.g., expressing CD34 and/or Thy1) and/or endothelial progenitor cells (e.g., expressing CD34 and VEGFR2) and/or neutrophils (identified morphologically and/or expressing e.g., CD10, CD14, CD31 and/or CD88) is assessed. A protein that reduces the level of the cell(s) compared to the level observed in the absence of the protein is considered to inhibit G-CSF signaling. In one example, the protein reduces the number of neutrophils without inducing neutropenia.

Other methods for assessing inhibition of G-CSF signaling are contemplated by the present disclosure.

### Visual Appearance

Pharmaceutical formulations encompassed by the present disclosure can be assessed for visual appearance to determine, for example, the colour and clarity or for the presence of visible particles.

### Dynamic Light Scattering

In one example, the particle size distribution is assessed using dynamic light scattering (DLS). DLS measures light scattered from particles based on Brownian motion and relies on differences in the index of refraction between the particle and the formulation. For example, the fluctuation of light intensity using a digital correlator is measured. The correlation functions are fitted into an analytical program (e.g., Malvern Zetasizer software) to calculate the particle size distribution. For the determination of Z-average hydrodynamic diameter, a cumulants analysis and the Stokes Einstein equation is performed using e.g., the viscosity of water (0.8872 mPa*s) at 25° C. The polydispersity index can also be obtained from the same cumulants analysis. Modality of fit is evaluated based on plots of size distribution versus intensity: modality can be described as monomodal (i.e., one peak) or multimodal (i.e., two or more peaks).

### Micro-Flow Imaging

In one example, sub-visible particles are assessed using micro-flow imaging (MFI). For example, digital images of particles suspended in a fluid are captured and automatically analysed for particle parameters, such as aspect ratio (AR) and intensity. The size (e.g., in μm) and count (i.e., number of particles per ml) can also be obtained. According to this method the data are morphologically categorised as proteinaceous (i.e., circular) and non-proteinaceous (i.e., non-proteinaceous particles such as air bubbles or silicone oil droplets) and a ratio of the non-proteinaceous particles to proteinaceous particles (i.e., the circular fraction) can be determined. A low circular fraction value indicates that the test article is comprised of mostly non-circular, likely proteinaceous particles.

### Size Exclusion Chromatography

In one example, aggregates/HMWS are assessed using size exclusion chromatography (SEC or SE-HPLC) which separates lower and higher molecular mass variants of the protein, as well as any impurities. According to this method, the results are described as the summation of aggregation peaks (APs) and summation of degradation peaks (DPs). For example, the identity of a pharmaceutical formulation of the present disclosure can be determined by comparing the chromatographic retention time of the major peaks with the retention time of the major peak of a reference standard.

### Differential Scanning Fluorimetry (DSF)

In one example, thermal stability of the pharmaceutical formulation of the present disclosure is assessed using differential scanning fluorimetry (DSF). DSF is a fluorescence-based assay using real-time PCR to monitor thermally induced protein denaturation by measuring changes fluorescence of a dye that binds preferentially to unfolded protein. For example, thermal unfolding and aggregation are monitored by changes in intrinsic protein fluorescence and static light scattering, respectively, as a function of temperature. According to this method, the midpoint of thermal transition (Tₘ) and onset of melting temperature (Tₒₙₛₑₜ) are determined by monitoring intrinsic fluorescence. The onset of aggregation temperature (T_{agg}) are determined by monitoring static light scattering, e.g., at 266 nm and 473 nm. Samples of the pharmaceutical formulation can be assessed across a range of temperatures, (e.g., 20° C.-95° C.) with a temperature increase at the rate of e.g., 0.5° C./min.

### Capillary Gel Electrophoresis

In one example, the pharmaceutical formulation of the present disclosure is assessed for stability and/or total accumulation of impurities using capillary gel electrophoresis (CGE). For example, both reduced-CGE (R-CGE) and non-reduced-CGE (NR-CGE) may be performed. In one example, R-GCE and NR-CGE are carried out using a capillary electrophoresis system (e.g., Beckman P/ACE MDQ or PA800) with a capillary length of e.g., 20.2 cm and 10 cm respectively from inlet to detection window, temperature control from e.g., 20 to 40° C. (±2° C.) and detector at e.g., 488 nm excitation.

### Cation Exchange Chromatography

In one example, the pharmaceutical formulation of the present disclosure is assessed for total charged variants (i.e. acid and basic species) using cation exchange (CEX) chromatography. CEX chromatography separates proteins according to their overall charge under native conditions. The CEX analysis is used to determine the purity of the product by separating the acidic and basic variants. The protein of interest must have a charge opposite to that of the functional group attached to the resin of the column in order to bind. Elution of the protein is achieved by increasing the ionic strength breaking the ionic interaction between the protein and the resin. The chromatographic technique separates the acidic, neutral and basic variants of a sample based on ionic strength. The peaks of interest are observed by UV detection at 280 nm where the acidic variants eluting first followed by neutral and basic variants. In one example CEX chromatography is carried out using a high performance liquid chromatography (HPLC) system (e.g. Dionex UltiMate 3000 BioRS (U) HPLC).

### Gibbs Free Energy (ΔG_{trend}; HUNK)

In one example, the chemical stability and aggregation behaviour of a pharmaceutical formulation of the present disclosure is evaluated by the change in the Gibbs free energy or ΔG_{trend} (HUNK) analysis. The ΔG_{trend} analysis measures the relationship between ΔG of protein unfolding and protein aggregation as a function of protein concentration. In the absence of aggregation, the ΔG of protein unfolding is a unimolecular process independent of protein concentration. If a change in ΔG is observed as a function of protein concentration, it signifies presence of aggregation. According to this method, there are two possible relationships between ΔG of protein unfolding and protein concentration if aggregation occurs:
1. ΔG_{trend} increases with protein concentration: This relationship indicates the presence of native state aggregation—the ΔG of protein unfolding increases (becomes more positive) as a function of protein concentration (i.e., concentration of native protein aggregates increases as a function of protein concentration); or
2. ΔG_{trend} decreases with protein concentration: This relationship indicates the presence of denatured state aggregation—the ΔG of protein unfolding decreases (become less positive) as a function of protein concentration (i.e., concentration of denatured protein aggregates increases as a function of protein concentration).

In a HUNK experiment the ΔG of protein unfolding is determined isothermally by measuring changes in a protein's intrinsic fluorescence spectrum (i.e., emission from tryptophan residues) as it unfolds in the presence of increasing amounts of denaturant.

In one example, ΔG_{trend} is determined by measuring ΔG of the protein unfolding at varying concentrations (e.g., 0.25, 0.6, 2.5, 6.0, 25.0 mg/ml) diluted to target concentration in a buffer of the pharmaceutical formulation of the disclosure. Each concentration level is titrated with increasing denaturant concentration (e.g., 32-point curve spanning urea concentration 2.00-8.74 M) while fluorescence spectra is measured from 300-500 nm (excitation 280 nm) with a slit width of 10 nm. The emission spectrum wavelength ratio of 350 nm/330 nm is plotted against urea concentration for each sample concentration level, and ΔG of protein unfolding determined using a 2 state (i.e., one transition) model fit. Determined ΔG values are plotted against sample concentration to determine ΔG_{trend}.

### Capillary Electrophoresis

In some examples, the formulation is assessed by capillary electrophoresis (CE). For example, the formulation may be assessed by capillary electrophoresis with sodium dodecylsulfate (CE-SDS) under non-reducing conditions to determine the proportion of LMWS present. Capillary electrophoresis is a separation method performed in submillimeter diameter capillaries and in micro- and nanofluidic channels. Proteins migrate through electrolyte solutions under the influence of an electric field. In the presence of SDS, proteins are denatured and are separated on the basis of their molecular weight. This enables the detection of LMWS present in the formulation, for example LMWS produced upon degradation (e.g., proteolytic degradation) of the protein.

### Turbidity Assessed by Absorbance at 550 nm

In one example, the turbidity of the pharmaceutical formulation of the present disclosure is assessed. For example, the turbidity is assessed using a spectrophotometer and measuring the absorbance at 550 nm.

### Syringeability

In one example, the syringeability of the pharmaceutical formulation of the present disclosure is assessed. For example, the formulation is expelled with a 2 ml syringe, 10 ml syringe, or left untreated as a pre-expulsion control. According to this method, the syringe plunger is pushed through the 2 ml syringes at a linear speed of 0.2 in/min and through the 10 ml syringes at 0.6 in/min until the plunger reaches the bottom and reaches the force of 30 N. Break-loose (BF) and glide (GF) forces are measured during expulsion and used to assess application suitability. Break-loose force describes the force required to initiate movement of the plunger (the initial 0.3 mm for 2 ml syringe and 0.5 mm for 10 ml syringe). Glide force Max refers to the maximum friction force required to sustain plunger movement. The maximum force value is measured from the end of the break loose region to the end of the glide force region (26 mm for 2 ml syringe and 24 mm for 10 ml syringe) prior to the point where the force reaches 30 N).

### Uses of the Pharmaceutical Formulation

As discussed herein, the present disclosure provides a method of treating or preventing a disease or condition in a subject, comprising administering a pharmaceutical formulation of the present disclosure to the subject. In one example, the present disclosure provides a method of treating or preventing a disease or condition in a subject in need thereof.

The present disclosure also provides for use of a pharmaceutical formulation of the present disclosure for treating or preventing a disease or condition in a subject comprising administering the pharmaceutical formulation of the present disclosure to the subject. In one example, the present disclosure provides for use of a pharmaceutical formulation of the present disclosure for treating or preventing a disease or condition in a subject in need thereof.

In some examples, the disease or condition is a neutrophil-mediated condition. In some examples, the neutrophil-mediated condition is an autoimmune disease, an inflammatory disease, cancer or ischemia-reperfusion injury.

Exemplary autoimmune conditions include autoimmune intestinal disorders (such as Crohn's disease and ulcerative colitis), arthritis (such as rheumatoid arthritis, psoriatic arthritis and or idiopathic arthritis, e.g., juvenile idiopathic arthritis) or psoriasis.

Exemplary inflammatory conditions include inflammatory neurological conditions (e.g., Devic's disease, a viral infection in the brain, multiple sclerosis and neuromyelitis optica), an inflammatory lung disease (e.g., chronic obstructive pulmonary disease [COPD], acute respiratory distress syndrome [ARDS] or asthma) or an inflammatory eye condition (e.g., uveitis).

In one example, the neutrophil-mediated condition is asthma.

In one example, the neutrophil-mediated condition is ARDS.

In one example, the neutrophil-mediated condition is ischemia-reperfusion injury. For example, the ischemia-reperfusion injury is due to or associated with tissue or organ transplantation (e.g., kidney transplantation). For example, the antibody is administered to a tissue or organ transplantation recipient, e.g., prior to organ collection and/or to a tissue or organ prior to transplantation or is administered to a harvested tissue or organ ex vivo.

In some examples, the neutrophil-mediated condition is psoriasis. In one example, the neutrophil-mediated condition is plaque psoriasis (also known in the art as “psoriasis vulgaris” or “common psoriasis”).

In one example, the neutrophil-mediated condition is a neutrophilic dermatosis or a neutrophilic skin lesion. For example, the neutrophilic dermatosis is a pustular psoriasis.

In one example, the neutrophilic dermatosis is selected from the group consisting of amicrobial pustulosis of the folds (APF); plaque psoriasis; CARD14-mediated pustular psoriasis (CAMPS); cryopyrin associated periodic syndromes (CAPS); deficiency of interleukin-1 receptor (DIRA); deficiency of interleukin-36 receptor antagonist (DIRTA); hidradenitis suppurativa (HS); palmoplantar pustulosis; pyogenic arthritis; pyoderma gangrenosum and acne (PAPA); pyoderma gangrenosum, acne, and hidradenitis suppurativa (PASH); pyoderma gangrenosum (PG); skin lesions of Behcet's disease; Still's disease; Sweet syndrome; subcorneal pustulosis (Sneddon-Wilkinson); pustular psoriasis; palmoplantar pustulosis; acute generalized exanthematic pustulosis; infantile acropustulosis; synovitis, acne, pustulosis; hyperostosis and osteitis (SAPHO) syndrome; bowel-associated dermatosis-arthritis syndrome (BADAS); neutrophilic dermatosis of the dorsal hands; neutrophilic eccrine hidradenitis; erythema elevatum diutinum; and Pyoderma gangrenosum. In one example, the neutrophilic dermatosis is hidradenitis suppurativa (HS) or palmoplantar pustulosis (PPP). The present disclosure also provides a method of reducing circulating neutrophils in a subject, the method comprising administering the formulation of the present disclosure. Such methods are useful in circumstances in which the subject is suffering from a disease or condition that is associated with neutrophils (e.g., neutrophil-mediated conditions).

In some examples, the subject is administered an effective amount of the protein in the formulation of the present disclosure. An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect treatment of a disease or condition as hereinbefore described. In some examples of the present disclosure, the term “effective amount” is meant an amount necessary to effect a change in a factor associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number. The effective amount can be administered in a single dose or in a dose repeated once or several times over a treatment period.

In some examples, the subject is administered a therapeutically effective amount of the protein in the formulation of the present disclosure. A “therapeutically effective amount” is at least the minimum concentration required to effect a measurable improvement of a particular disease or condition. A therapeutically effective amount herein may vary according to factors such as the disease state, age, sex, and weight of the patient, and the ability of the antibody or antigen binding fragment thereof to elicit a desired response in the individual. A therapeutically effective amount is also one in which any toxic or detrimental effects of the protein are outweighed by the therapeutically beneficial effects.

In one example, the pharmaceutical formulation of the present disclosure is administered to the subject in an amount to reduce the severity of the disease or condition in the subject.

In one example, the subject is at risk of developing a neutrophil-mediated condition. A subject is at risk if he or she has a higher risk of developing a neutrophil-mediated condition than a control population. The control population may include one or more subjects selected at random from the general population (e.g., matched by age, gender, race and/or ethnicity) who have not suffered from or have a family history of a neutrophil-mediated condition. A subject can be considered at risk for a disease or condition if a “risk factor” associated with a neutrophil-mediated condition is found to be associated with that subject. A risk factor can include any activity, trait, event or property associated with a given disorder, for example, through statistical or epidemiological studies on a population of subjects. A subject can thus be classified as being at risk for a neutrophil-mediated condition even if studies identifying the underlying risk factors did not include the subject specifically.

In one example, the subject is at risk of developing a neutrophil-mediated condition and the pharmaceutical formulation of the present disclosure is administered before or after the onset of symptoms of a neutrophil-mediated condition. In one example, the pharmaceutical formulation is administered before the onset of symptoms of a neutrophil-mediated condition. In one example, the pharmaceutical formulation is administered after the onset of symptoms of a neutrophil-mediated condition. In one example, the pharmaceutical formulation of the present disclosure is administered at a dose that alleviates or reduces one or more of the symptoms of a neutrophil-mediated condition in a subject at risk.

The methods of the present disclosure can be readily applied to any form of a neutrophil-mediated condition in a subject. In one example, a method of the disclosure reduces any symptom of a neutrophil-mediated condition known in the art and/or described herein. As will be apparent to the skilled person a “reduction” in a symptom of a disorder in a subject will be comparative to another subject who also suffers from a disorder but who has not received treatment with a method described herein. This does not necessarily require a side-by-side comparison of two subjects. Rather population data can be relied upon. For example, a population of subjects suffering from a neutrophil-mediated condition who have not received treatment with a method described herein (optionally, a population of similar subjects to the treated subject, e.g., age, weight, race) are assessed and the mean values are compared to results of a subject or population of subjects treated with a method described herein.

A method of the present disclosure may also include co-administration of the pharmaceutical formulation according to the disclosure together with the administration of another therapeutically effective agent for the prevention or treatment of a neutrophil-mediated condition.

In one example, the pharmaceutical formulation of the disclosure is used in combination with at least one additional known compound or therapy which is currently being used or is in development for preventing or treating neutrophil-mediated condition, or reducing circulating neutrophils. For example, the other compound is an anti-inflammatory compound, e.g, methotrexate or a non-steroidal anti-inflammatory compound. Alternatively, or additionally, the other compound is an immunosuppressant. Alternatively, or additionally, the other compound is a corticosteroid, such as prednisone and/or prednisolone. In on example, the other compound is methotrexate. Alternatively, or additionally, the other compound is cyclophosphamide.

In some examples, the formulation is administered in combination with a cell. In some examples, the cell is a stem cell, such as a mesenchymal stem cell.

In some examples, the formulation is administered in combination with a gene therapy.

In some examples, the formulation is administered in combination with a non-pharmaceutical intervention, for example, apharesis, such as plasmapheresis, cytapheresis, leukapheresis, granulocyte and/or monocyte apheresis. In this context, the formulation can be administered during the period of time in which the non-pharmaceutical intervention is being performed and will be considered “in combination with” the non-pharmaceutical intervention. For example, the non-pharmaceutical intervention may be granulocyte and/or monocyte apheresis, which is performed once per week for five weeks and the formulation can be administered over this time period. In one example, the formulation is administered before the non-pharmaceutical intervention. In one example, the formulation is administered after the non-pharmaceutical intervention.

Another non-pharmaceutical intervention is light therapy. Light therapy is used to treat some neutrophilic dermatoses.

As will be apparent from the foregoing, the present disclosure provides methods of concomitant therapeutic treatment of a subject, comprising administering to a subject in need thereof an effective amount of a first agent and a second agent or therapy, wherein the first agent is a pharmaceutical formulation of the present disclosure, and the second agent or therapy is also for the prevention or treatment of a neutrophil-mediated condition.

As used herein, the term “concomitant” as in the phrase “concomitant therapeutic treatment” includes administering a first agent in the presence of a second agent or therapy. A concomitant therapeutic treatment method includes methods in which the first, second, third or additional agents/therapies are co-administered. A concomitant therapeutic treatment method also includes methods in which the first or additional agents are administered in the presence of a second or additional agent or therapy, wherein the second or additional agent or therapy, for example, may have been previously administered. A concomitant therapeutic treatment may be executed step-wise by different actors. For example, one actor may administer to a subject a first agent and as a second actor may administer to the subject a second agent or therapy and the administering steps may be executed at the same time, or nearly the same time, or at distant times, so long as the first agent (and/or additional agents) are after administration in the presence of the second agent or therapy (and/or additional agents or therapies). The actor and the subject may be the same entity (e.g. a human).

### Kits and Other Compositions of Matter

Another example of the disclosure provides kits containing a pharmaceutical formulation of the present disclosure useful for the treatment or prevention of a disease or condition as described above.

In one example, the kit comprises (a) a container comprising a pharmaceutical formulation of the present disclosure; and (b) a package insert with instructions for treating or preventing a disease or condition in a subject.

In one example, the kit comprises (a) at least one pharmaceutical formulation of the present disclosure; (b) instructions for using the kit in treating or preventing the disease or condition in the subject; and (c) optionally, at least one further therapeutically active compound or drug.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for treating a neutrophil-mediated condition and may have a sterile access port (for example, the container may be an intravenous solution bag or a vial having a stopper pierceable by a hypodermic injection needle). The label or package insert indicates that the composition is used for treating a subject eligible for treatment, e.g., one having or predisposed to developing a neutrophil-mediated condition, with specific guidance regarding dosing amounts and intervals of the pharmaceutical formulation and any other medicament being provided. The kit may further include other materials desirable from a commercial and user standpoint, including filters, needles, and syringes. In some examples of the present disclosure, the formulation can be present in an injectable device (e.g., an injectable syringe, e.g., a prefilled injectable syringe). The syringe may be adapted for individual administration, e.g., as a single vial system including an autoinjector (e.g., a pen-injector device). In one example, the injectable device is a prefilled pen or other suitable autoinjectable device, optionally with instruction for use and administration.

The kit optionally further comprises a container comprising a second medicament, wherein the pharmaceutical formulation is a first medicament, and which article further comprises instructions on the package insert for treating the subject with the second medicament, in an effective amount. The second medicament may be a therapeutic protein set forth above.

In one example, the disclosure provides a prefilled syringe or autoinjector comprising a formulation of the present disclosure. In one example, the prefilled syringe is a glass luer syringe with plunger.

In one example, the disclosure provides a vial comprising a formulation of the disclosure.

The present disclosure includes the following non-limiting Examples.

### EXAMPLES

### Example 1: Materials and Methods

The materials used for the following Examples, their catalogue numbers and the suppliers are listed in Table 1.

TABLE 1
Materials used for the examples

| | Catalogue | |
|---|---|---|
| Material | Number | Supplier |
| 2 mL Glass Vials, | 1551745 | Schott Igar glass |
| 13 mm neck | | |
| 13 mm Stopper | 19700004 | West |
| 13 mm cap | 54133014 | West |
| 5 mL Biocontainer | 2035-0005 | Nalgene |
| 10 mL Biocontianer | 2035-0010 | |
| 20 mLBiocontainer | 2035-0020 | |
| 30 mL Biocontainer | 342020-0030 | |
| 0.5 mL vials | MAT3750 | Thermo Scientific |
| Sepra Seals | MAT4464 | Matrix |
| Slide-A-Lyzer ™ Dialysis | 87731 | Thermo Scientific |
| Cassette | | |
| Amicon-Ultra- | UFC903024 | Merck |
| 15CentrifugalFilter Device | | |
| (30 kDa) | | |
| Pellicon ® 3 with Ultracel ® | P3C030D00 | Merck |
| 30 kDa membrane, D | | |
| screen, 88 cm | | |
| Millex-GP Syringe Filter | SLGP033RS | Merck |
| Unit, 0.22 μm | | |
| Millipore Steritop, | S2GPT10RE | Merck |
| 0.22 μm | | |
| Hydrochloric acid, 6M | 1.10164 | Merck |
| Sodium hydroxide | 1048 | ACR Chemical |
| solution, 4M | | Reagents |
| 5M Sodium Hydroxide | 1.37041 | Merck |
| L-Histidine | 1.04352 | Merck |
| Sodium chloride | 1.06400 | Merck |
| L-Proline | 1.07430 | Merck |
| L-Arginine•HCl | 1.01544 | Merck |
| L-Methioine | M8439 | Sigma Aldrich |
| Polysorbate 80 | HX2 | NOF Corporation |

### Preparation of Formulations

The bulk anti-G-CSFR antibody material was buffer exchanged into the required formulations & pHs via either dialysis cassettes, centrifugation or TFF (˜7 buffer exchanges cycles) before final concentration in excess of the target concentration (target >150 mg/mL) and recovered. The protein concentration was measured, surfactant added to the target concentration, and all formulations diluted to the target protein concentration with the formulation diluent. If maximum concentration was below the target no further dilution was performed. Formulations were 0.2 μm filtered and stored in Biocontainers (Nalgenes) or glass vials at various different fill volumes.

### Visual Appearance

Visual appearance was conducted in an inspection station equipped with a white and black background and fluorescent light. Formulations in vials were gently swirled without producing bubbles then inspected for colour, clarity and the presence of visible particles. Inspections were conducted by two independent inspectors.

### pH Measurements

The pH of the formulations was measured using a Mettler Toledo SevenExcellence pH meter equipped with a InLab®Ultra Micro ISM electrode.

### UV Spectroscopy

Protein concentration was measured by using A280/UV determination on the formulations via two methods:
neat on an IMPLEN P360 Nanophotometer Measurements were conducted in triplicate and the mean value of the measurement calculated
via gravimetric dilution to on the Shimadzu UV-1700 Spectrophotometer and performed in duplicate.

Size exclusion chromatography (SEC)-high performance liquid chromatography (HPLC)

SEC-HPLC was used to determine the protein aggregation profile of the formulations. Intact protein was detected at 280 nm with monomer species, high molecular weight species (HMWS, aggregates) and low molecular weight species (LMWS, fragments) reported as a relative area %. Internal and external references were used to validate the run. This was performed with a Dionex system (Ultimate 3000) via two methods:
(i) first method was performed with an Acquity BEH200 column (Waters, 1.7 μm, 4.6×150 mm) to analyse the samples. Samples were diluted to 5 g/L in appropriate buffer, 3 μL was injected or 10 g/L in appropriate buffer and 1.5 μL was injected. Separation was performed under isocratic conditions at a flow rate of 0.3 mL/min. Mobile phase consisted Bis-Tris Propane buffer (pH 7.0) with a run time of 12 min.
(ii) second method was equipped with a TSkgel G3000SWxL column (TOSOH, 5 μm, 7.8×300 mm 250A) to analyse the samples. Samples were diluted to 5 g/L in appropriate buffer, 10.0 μL was injected and the separation was performed under isocratic conditions at a flow rate of 1.0 mL/min. Mobile phase consisted of sodium phosphate buffer (pH 7.0) with a run time of 15 min.

### Cation Exchange Chromatography (CEX)

CEX-HPLC was used to determine the proportions of proteinaceous acidic, main and basics species. A Dionex system (Ultimate 3000) equipped with a Waters Acquity ProteinPak™ HiRes CM 7 μm 4.6×100 mm column was used to analyse the samples. Samples were diluted to 10 g/L in appropriate buffer, 2.5 μL injection volume &/or 5 g/L in appropriate buffer, 5 μL injection volume was used and separation was conducted with a gradient method at 0.7 ml/min. Briefly, two aqueous IVIES buffers at pH 6.2 with an increasing salt gradient over a run period of 24 minutes. Species were detected at 280 nm, identified against a reference standard and reported as relative Area percentage over the integrated area.

### Osmolality Measurements

Osmolality of the formulations was measured by using a Vapro 5600 vapour pressure osmometer. Sample volumes were 10 μL. Measurements were conducted in triplicate and the mean values of the measurements calculated.

### pH Measurement

pH was measured using a Mettler Toledo SevenExcellence pH meter equipped with a InLab®Ultra Micro ISM electrode

### Analysis of Polysorbate 80 (PS80)

RP-HPLC was used to quantify the amount of PS80 at the initial time point (TO) in the different formulations. PS80 standard and the samples were treated with ethanol followed by 0.1M KOH at 40° C. followed by sample analysis of oleic acid resulting from hydrolysis by a reverse phase HPLC method. A Dionex (Ultimate 3000) System (or equivalent) equipped with a Nova-Pak® C18 3.9×150 mm, 4 μm reverse phase column (Waters) was used to analyze the samples. Injection volume was 15 μL and separation was conducted using an isocratic method at 2.0 ml/min. Mobile phase was 80% acetonitrile with 20% potassium dihydrogen phosphate buffer at pH 2.8. Column temperature was set to 40° C. Species were detected at 250 nm, and quantified using a standard calibration curve generated by the PS80 standard solutions. Data is reported as % (w/v) of PS80.

### Capillary Gel Electrophoresis (CGE),

The protein “banding pattern” was obtained by Capillary Gel Electrophoresis. Analysis was performed using a microfluidic LabChip GXII system (Perkin Elmer Australia Pty Ltd) or PA800 (Beckman Coulter). The protein electrophoresis on the microfluidic chip was achieved by integration of the main features of one-dimensional SDSPAGE: these include the separation, staining, de-staining, and detection. Denatured proteins were loaded onto the chip directly from a microtiter plate through a capillary sipper. The samples were then electrokinetically loaded and injected into the 14 mm long separation channel containing a low viscosity matrix of entangled polymer solution. The entire sample preparation procedures were performed according to the manufacturers protocol. For non-reducing samples, protein solution were diluted to 2 g/L with non-reducing buffer and Milli-Q water. Reducing samples were diluted with kit buffer containing DTT. Denaturation occurred at 40° C. for 20 min for non-reduced samples and at 80° C. for 15 min for reduced samples. The PA800 method separates protein species based on their molecular weight, and detection occurs using a UV detector at 214 nm. Under non-reducing conditions, prior to analysis the sample is denatured by addition of Sodium Dodecyl Sulphate (SDS) and heat, followed by alkylation of free cysteines using N-ethylmaleimide (NEM). The relative main peak (purity) and low molecular weight species (LMWS; impurity) are measured. Under reducing conditions, prior to analysis the sample is denatured by addition of SDS and heat, followed by reduction of disulphide bonds with β-mercaptoethanol (BME). Results were reported in relative area percentage for LMWS Intact and HMWS for non-reduced samples. For reduced samples, heavy and short chain fractions were considered.

### Sub-Visible Particle Count Testing

Sub-visible particle counting was performed by Light obscuration using HIAC 9703+ utilising a low volume method of 4×1 mL, with the average of the final 3 runs being calculated and reported as particles ≥2 μm, ≥5 μm, ≥10 μm and ≥25 μm. Analysis of sub-visible particles morphology, size distribution and counts was also performed using a FlowCam Biologics instrument (a Dynamic/flow Imaging Particle Analysis—DIPA-technique) on selected formulations of interest. A minimum sample volume of 0.5 mL was used. Measurements were conducted in triplicate per formulation and the mean values of the measurements calculated and particles counted as 2 to 5 μm, 5 to 10 μm, 10 to 25 μm and >25 μm.

### Reverse Phase HPLC

A RP-HPLC method was used to determine the total amount of oxidized species as a percentage of the total area, and the relative amount of oxidation of the HC FC/2, Light Chain region and HC Fd′ domain. The sample is initially diluted with PBS to 10 mg/ml. The sample is digested with IdeS enzyme (Genovis FabRICATOR), which performs a site-specific cleavage below the hinge region of the IgG followed by an incubation step of an hour at 37° C. This is followed by denaturation and reduction of the sample with the addition of 20 mM DTT, 1 mM EDTA, 100 mM MES, pH 5.5, 3 M Guanidine-HCl and an incubation of 30 minutes at 56° C. The sample is then diluted with 25:75 v/v sample: MPA (0.1% TFA) to adjust the pH of the sample to enhance the sample stability. A Thermo Ultimate 3000 (or equivalent) equipped with an Acquity UPLC BEH300 C4 1.7 μm, 50 mm×2.1 mm column was used to analyse the samples. A target loading of 5 μg was used with the final sample concentration and separation was conducted with a gradient method at 0.30 ml/min. Column temperature was set to 70° C. Briefly, two buffers (0.1% trifluoroacetic acid in water and 0.08% TFA in acetonitrile) were alternated over a period of 30 minutes. Species were reported at 280 nm, identified against a reference standard and reported as Relative Area percentage over the integrated area. The chromatograms of the samples contain three main peaks of light chain (LC), Fd′ and monomeric Fc (Fc/2), the respective oxidation products associated with each domain elutes slightly earlier than each of the main peaks listed. The oxidised species for each domain is reported separately, as the percentage area relative to the area of the total peaks in that domain.

### Closed Container Integrity

The closed container integrity of the formulations in the vials is performed via vacuum decay method using the glass vial VeriPac 455.

### Endotoxin

A limulus amebocyte lysate method is used to measure endotoxin by the kinetic chromogenic method. Samples were required to be tested at 4 different dilutions, increasing by 10 fold and results reported from the valid result which has achieved an end-point result and has a PPC recovery of at closest to 100%.

### Potency

The potency ELISA measures in vitro protein binding of CSL324 to its target G-CSF-R. A 96-well microtitre plate is coated with GCSF-R at a fixed concentration, after which CSL324 antibody at a range of concentrations is added. The plate is washed, and the remaining bound CSL324 antibody is detected by means of horseradish peroxidase (HRP) conjugated IgG. Colour development of the HRP substrate is measured in a plate reader at 450 nm, and the data is fitted using a 4-parameter logistic (4PL) regression model. Relative potency is then calculated using parallel line analysis against the reference standard, and the result is reported as percent relative to reference standard.

### Example 2: Stabiliser Components

The aim of the experiments described in the following examples was to produce a formulation of CSL324, an antibody that binds to GCSF-R, which had long term stability and was suitable for subcutaneous administration. The starting formulation contained 10 mg/mL CSL324, 20 mM histidine buffer at pH 6.4, 140 mM NaCl, and 0.02% w/w PS80.

As an initial step, the stability, osmolality, and viscosity of four formulations of 130-150 mg/mL CSL324, each comprising different stabiliser components, was evaluated. Stability was assessed by measuring the percentage of high molecular weight species (HMWS) present after 4 months of storage at 5° C. by SE-HPLC. Each of the four formulations comprised 20 mM histidine at pH 6.4 or 5.5 and 0.02% polysorbate 80, which were present in the starting formulation.

Table 2 shows that the NaCl-containing formulations had higher percentage of HMWS and viscosity compared to the other formulations after storage. The NaCl-containing formulations also had higher opalescence and lower thermal stability.

TABLE 2
Stability, osmolality, and viscosity of CSL324 formulations
comprising different stabiliser components

| | | % HMWS | | |
|---|---|---|---|---|
| | | by SE-HPLC | | Viscosity |
| Stabiliser | | (4 months at | Osmolality | at 20° C. |
| components | pH | 5° C.) | (mOsm/kg) | (mPa*s) |
| 140 mM NaCl | 6.4 | 3.1 | 296 | 12.2 |
| 140 mM proline, | 6.4 | 2.8 | 451 | 7.6 |
| 150 mM arginine | | | | |
| 260 mM proline, | 6.4 | 2.7 | 331 | 10.9 |
| 7.5 mM methionine | | | | |
| 100 mM arginine, | 6.4 | 2.9 | 319 | 9.6 |
| 50 mM NaCl | | | | |
| 140 mM NaCl | 5.5 | 1.7 | 298 | 8.7* |
| 140 mM proline, | 5.5 | 1.6 | 461 | 10.8 |
| 150 mM arginine | | | | |
| 260 mM proline, | 5.5 | 1.5 | 327 | 13.1 |
| 7.5 mM methionine | | | | |
| 100 mM arginine, | 5.5 | 1.6 | 314 | 7.9* |
| 50 mM NaCl | | | | |

*NaCl containing formulations could only be concentrated to a maximum of 132 mg/mL

Predictive analyses were also performed on the above formulations to determine which stabiliser components gave the most favourable solute-solvent interactions. It was found that the best attributes were associated with the formulations without NaCl. It was therefore concluded that NaCl was not a suitable stabiliser for formulations of high concentrations of CSL324. The stabilisers, proline and arginine, and antioxidant methionine were thus chosen for further optimisation.

The effects of proline and arginine were assessed in two formulations of 150 mg/mL CSL324 (containing 20 mM histidine buffer with a target pH of 6.4 and 0.03% w/w polysorbate 80) in relation to pH, viscosity, and stability. Table 3 shows the results of the analyses.

TABLE 3
Effects of proline and arginine in 150 mg/mL CSL324 formulations

| | | | Viscosity |
|---|---|---|---|
| | | | at 20° C. |
| Stabiliser component | Target pH | Actual pH | (mPa*s) |
| 260 mM proline | 6.4 | 6.6 | 19 |
| 95 mM proline, 100 mM arginine | 6.4 | 6.4 | 10 |

Table 3 shows that high concentrations of proline were found to interfere with the pH of the formulations, and had higher viscosity, when present as a lone stabiliser. Reducing the proline concentration to 95 mM and adding 100 mM arginine significantly reduced the viscosity of the formulation and resulted in an actual pH equivalent to the target pH. There were no significant differences in percentages of HMWS, LMWS, acidic and basic variants following 12 weeks storage at 5° C.

The amounts of proline and arginine in the formulation were further optimised. Two formulations of 150 mg/mL CSL324 (containing 20 mM histidine buffer with target pH of 6.4 and 0.02% w/w polysorbate 80) were compared in relation to their osmolality, viscosity and stability. Stability was assessed by measuring the percentage of HMWS, LMWS and acidic and basic variants present after 12 weeks storage at 35° C. Table 4 shows the results of the analyses.

TABLE 4
Optimisation of proline and arginine concentrations

| | | Viscosity | % HMWS | | % acidic | % basic |
|---|---|---|---|---|---|---|
| Stabiliser | Osmolality | at 20° C. | by | % LMWS | species by | species by |
| component | (mOsm/kg) | (mPa*s) | SE-HPLC | by Caliper | CE-HPLC | CE-HPLC |
| 140 mM proline, | 465 | 10 | 4.4 | 6.1 | 48 | 14 |
| 150 mM arginine | | | | | | |
| 95 mM proline, | 326 | 11 | 4.5 | 5.9 | 51 | 13 |
| 100 mM arginine | | | | | | |

Table 4 demonstrates that the formulation comprising 95 mM proline and 100 mM arginine had lower osmolality after storage at 35° C. for 12 weeks, relative to the formulation comprising 140 mM proline and 150 mM arginine. There were no large differences observed for viscosity or percentage of HMWS, LMWS, acidic species and basic species. These results show that the proline and arginine levels could be reduced to near 100 mM, giving lower osmolality without any loss of stability.

### Example 3: Antioxidants and Surfactants

### Antioxidant

To further optimise the CSL324 formulation, the effects of methionine, as a potential antioxidant, were assessed. Four formulations of 150 mg/mL CSL324 (containing 20 mM histidine buffer with target pH of 6.4 and 0.02 w/w polysorbate 80) were compared in relation to their stability following two weeks storage at 35° C. Table shows the results of the analyses.

TABLE 5
Effect of methionine on stability
of 150 mg/mL CSL324 formulations

| | % HMWS | % LMWS | % HC | % acidic | % basic |
|---|---|---|---|---|---|
| Stabiliser | by | by | Fd′ by | species | species |
| component | SE-HPLC | Caliper | RP-HPLC | by CEX | by CEX |
| 140 mM NaCl | 3.4 | 1.5 | 12.0 | 21 | 14 |
| 140 mM NaCl, | 3.2 | 1.4 | 11.8 | 21 | 14 |
| 7.5 mM | | | | | |
| methionine | | | | | |
| 260 mM | 3.0 | 1.5 | 12.3 | 22 | 14 |
| proline | | | | | |
| 260 mM | 2.9 | 1.5 | 12.3 | 22 | 14 |
| proline, | | | | | |
| 7.5 mM | | | | | |
| methionine | | | | | |

Table 5 demonstrates that there were no improvements in stability of the formulations comprising methionine compared to the equivalent formulation without methionine. Also, there was no significant difference in stability observed following peroxide (0.1% hydrogen peroxide, 25° C., 5 hours) and UV light (0.5×ICH, 3 days, 25° C.) stress between the formulations with and without methionine.

The effects of methionine were also assessed in a formulation comprising 50, 100 or 150 mg/mL CSL324, 20 mM histidine (pH 6.4), 95 mM proline and 100 mM arginine. Stability of the formulations with and without methionine was assessed after 12 weeks storage at 5, 25 and 35° C. There was no significant difference in any of percentage of HMWS, LMWS, basic or acidic variants observed between the formulation with and without methionine.

### Surfactant

To further optimise the CSL324 formulation, the effects of polysorbate 80 were assessed in relation to protection against stirring stress and particulate formation after dilution with saline to 0.2 mg/mL. Four concentrations of polysorbate 80 were assessed (0.02%, 0.05%, 0.1% and 0.3%) in a formulation comprising 150 mg/mL CSL324 in 20 mM histidine (pH 6.4) and 140 mM NaCl.

0.02% w/v polysorbate 80 was sufficient to provide adequate protection against stirring stress induced by stirring at 130 rpm for 60 min. However, it was observed that increased concentrations of polysorbate 80 provided better protection against particulate formation following inversions after dilution with saline to 0.2 mg/mL CSL324 (Table 6). It was therefore decided to increase the concentration of polysorbate from 0.02% in the starting formulation to 0.03% w/v.

TABLE 6
effect of polysorbate 80 on dilution with saline

| | Dynamic Imaging Particle Analysis by FlowCam Particles/mL | | | | | | | | | |
|---|---|---|---|---|---|---|---|---|---|---|
| | Initial | | | | | 5 h at 25° C. | | | | |
| Predilution | 2-5 | 5-10 | 10-25 | >25 | | 2-5 | 5-10 | 10-25 | >25 | |
| [PS80] | μm | μm | μm | μm | Total | μm | μm | μm | μm | Total |
| 0.02% | 238 | 218 | 142 | 45 | 643 | 112 | 110 | 62 | 40 | 324 |
| 0.05% | 153 | 102 | 96 | 30 | 381 | 121 | 57 | 27 | 9 | 214 |
| 0.1% | 101 | 75 | 64 | 7 | 246 | 60 | 42 | 35 | 4 | 141 |
| 0.3% | 163 | 71 | 31 | 9 | 274 | 344 | 172 | 148 | 0 | 663 |

### Example 4: Optimisation of pH

The effect of differing pH on stability of the CSL324 formulations was assessed. Initially, four formulations of 150 mg/mL CSL324 (containing 20 mM histidine buffer with target pH of 6.4 or 5.5) were assessed in relation to their effect on aggregation following storage at 5° C. for four months. Table 7, below, demonstrates that a pH of 5.5 resulted in less aggregation for all four of the formulations tested relative to pH 6.4.

TABLE 7
Effect of pH on stability of 150 mg/mL CSL324 formulations

| | % HMWS by | % HMWS by |
|---|---|---|
| | SE-HPLC | SE-HPLC |
| | (change from initial) | (change from initial) |
| Stabiliser components | at pH 6.4 | at pH 5.5 |
| 140 mM NaCl | 2.2 | 0.9 |
| 140 mM proline, | 1.8 | 0.8 |
| 150 mM arginine | | |
| 260 mM proline, | 1.7 | 0.7 |
| 7.5 mM methionine | | |
| 100 mM arginine, | 2.0 | 0.8 |
| 50 mM NaCl | | |

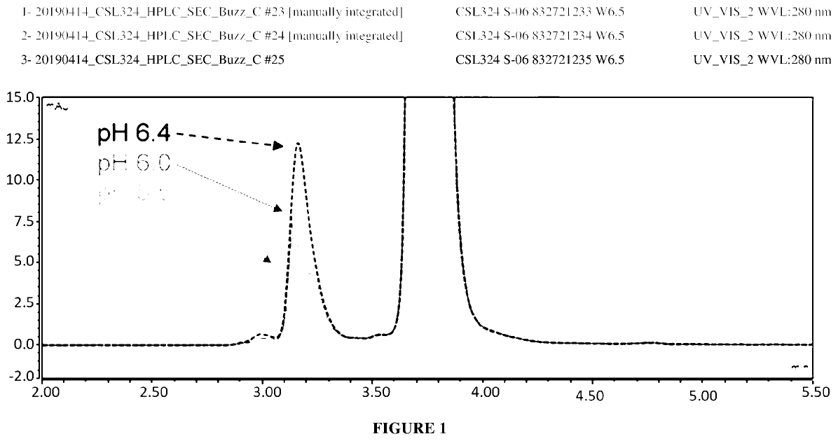

The effect of differing pH on stability of a CSL324 formulation comprising 150 mg/mL CSL324, 20 mM histidine buffer (with a pH of 6.4, 6.0 or 5.5), 95 mM proline and 100 mM arginine was also assessed. The level of aggregation was assessed by SE-HPLC following storage at 25° C. for 6.5 weeks. FIG. 1 demonstrates that the amount of HMWS decreased as the pH of the formulation decreased.

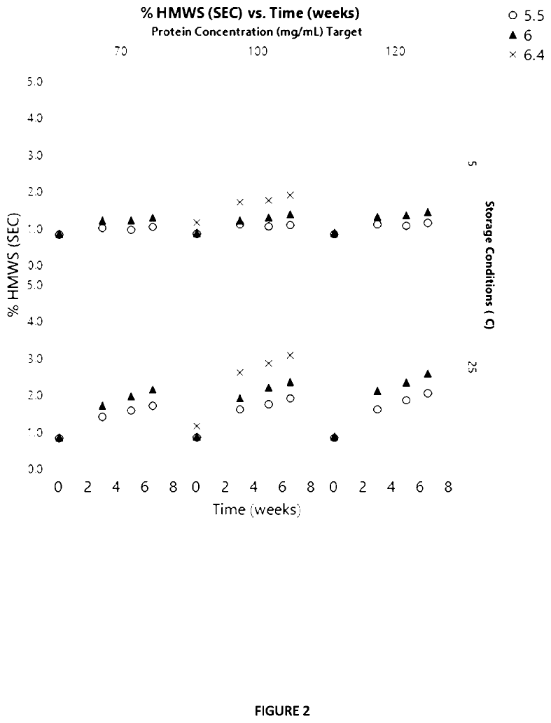

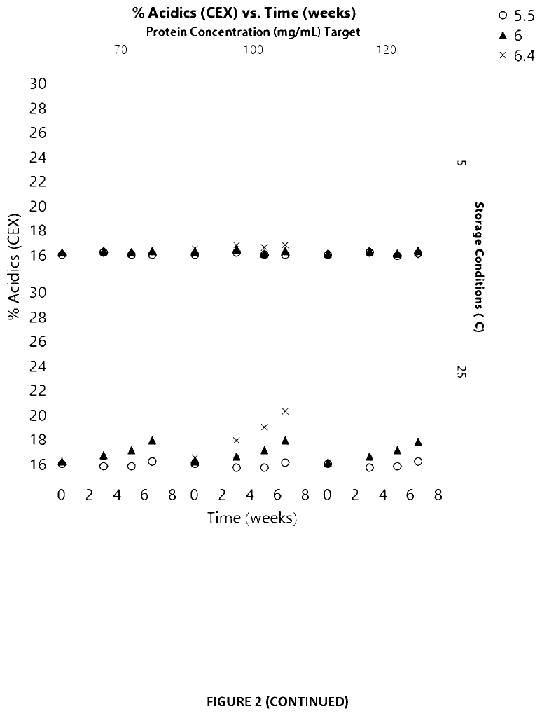

The effect of pH was also assessed over a time course of eight weeks in relation to HMWS and amount of acidic species produced during storage at 5° C. or 25° C. Formulations comprising 120, 100, or 70 mg/mL CSL324, 20 mM histidine buffer (with a pH of 6.4, 6.0 or 5.5), 95 mM proline and 100 mM arginine were tested. FIG. 2A shows that higher percentage of HMWS was observed at a pH of 6.0 or 6.4 relative to pH 5.5, for all protein concentrations tested. Similarly, FIG. 2B shows that there were larger increases in acidic species (and corresponding decrease in main species), as determined by cation exchange chromatography, over time at higher pH for all protein concentrations tested.

The above formulations were stored for up to 9 months at 5° C. or 21 weeks at 25° C. The effects of pH on the stability of these formulations are summarised below in Table 8 (100 mg/mL CSL324).

TABLE 8
effects of pH on long term storage (% change from initial quantity shown)

| | % HMWS by | % monomer | % LMWS by | % acidic species |
|---|---|---|---|---|
| | SE-HPLC | by SE-HPLC | Caliper | by CE-HPLC |
| Protein conc., | (change from | (change from | (change from | (change from |
| pH, temp | initial) | initial) | initial) | initial) |
| 120 mg/mL, pH | 1.0 | −1.0 | −0.1 | 0 |
| 5.5, 5° C. | | | | |
| 120 mg/mL, pH | 1.7 | −1.7 | 0.0 | −1 |
| 6.0, 5° C. | | | | |
| 100 mg/mL, pH | 0.9 | −1.0 | 0.1 | 0 |
| 5.5, 5° C. | | | | |
| 100 mg/mL, pH | 1.6 | −1.6 | 0.0 | −1 |
| 6.0, 5° C. | | | | |
| 100 mg/mL, pH | 1.9 | −1.9 | 0.2 | −2 |
| 6.4, 5° C. | | | | |
| 70 mg/ml, pH | 0.8 | −0.8 | 0.0 | 0 |
| 5.5, 5° C. | | | | |
| 70 mg/mL, pH | 1.4 | −1.4 | 0.1 | −1 |
| 6.0, 5° C. | | | | |
| 120 mg/mL, pH | 2.2 | −2.2 | 1.8 | −3 |
| 5.5, 25° C. | | | | |
| 120 mg/mL, pH | 2.5 | −2.5 | 2.0 | −7 |
| 6.0, 25° C. | | | | |
| 100 mg/mL, pH | 2.0 | −2.1 | 1.7 | −3 |
| 5.5, 25° C. | | | | |
| 100 mg/mL, pH | 2.4 | −2.5 | 2.0 | −7 |
| 6.0, 25° C. | | | | |
| 100 mg/mL, pH | 2.6 | −2.6 | 2.5 | −12 |
| 6.4, 25° C. | | | | |
| 70 mg/ml, pH | 1.8 | −1.9 | 1.8 | −3 |
| 5.5, 25° C. | | | | |
| 70 mg/mL, pH | 2.2 | −2.2 | 2.0 | −7 |
| 6.0, 25° C. | | | | |

These results indicate that the formulations of CSL324 were stable at all pH's tested (5.5, 6.0, 6.4). However, the formulations were most stable at pH 5.5.

### Example 5: Exemplary Formulation

An exemplary antibody formulation, based on the results described above, is shown in Table 9.

TABLE 9
Exemplary antibody formulation

| Component | Amount or concentration | |
|---|---|---|
| Antibody | 120 | mg/ml |
| L-Histidine | 20 | mM |
| L-Arginine | 100 | mM |
| L-Proline | 100 | mM |
| Polysorbate 80 | 0.03% | w/v |
| pH | 5.7 | (5.5-5.9) |
| Osmolality (mOsm/kg) | ~315 | mOsm/kg |
| | (280 to 350 | mOsm/kg) |

| | |
|---|---|
| Viscosity | 5.4 mPa*s (@ 20° C.), |
| | 4.6 mPa*s (@ 25° C.) |

While the concentration of antibody exemplified in Table 9 is 120 mg/mL, the formulation is also suited to lower concentrations of antibody.

### Example 6: Long-Term Stability

The long-term stability of the formulation provided in Table 9 was assessed by holding the formulation at 5° C. (±3° C.) for 24 months or 25° C. (±2° C.) for 18 months. The results are shown in Tables 10 and 11.

TABLE 10
Long-term stability of exemplary CSL324 formulation after 24 months at 5° C. ± 3° C.

| | | | Number of months | | | | | | |
|---|---|---|---|---|---|---|---|---|---|
| Test | Units | Provisional acceptance criteria | 0 | 3 | 6 | 9 | 12 | 18 | 24 |
| Particle count (LO) | No. of | ≤6,000 particles of ≥10 μm | 12 | 35 | 8 | 91 | 40 | 9 | 27 |
| average cumulative | particles/ | ≤600 particles of ≥25 μm | 1 | 0 | 1 | 11 | 5 | 0 | 5 |
| counts/container | container | | | | | | | | |
| Description | N/A | Opalescent to clear, yellow to colourless | Pass | Pass | Pass | Pass | Pass | Pass | Pass |
| | | liquid. No visible particles. | | | | | | | |
| pH | pH units | 5.5-5.9 | 5.6 | 5.6 | 5.6 | 5.6 | 5.6 | 5.6 | 5.6 |
| Protein concentration | mg/mL | 110-130 mg/ml | 121 | 121 | 121 | 121 | 121 | 120 | 121 |
| by UV | | | | | | | | | |
| spectrophotometry | | | | | | | | | |
| (A280 nm) | | | | | | | | | |
| SE-HPLC | % peak area | ≤5.0% HMWS | 0.7 | 1.4 | 1.8 | 2.1 | 2.5 | 2.8 | 3.0 |
| | | ≥95.0% monomer | 99.2 | 98.6 | 98.2 | 97.9 | 97.5 | 97.2 | 97.0 |
| | | 10-30% acidic species | 16 | 16 | 16 | 16 | 17 | 17 | 17 |
| CEX-HPLC | % peak area | 50-80% main peak | 69 | 70 | 70 | 69 | 69 | 69 | 68 |
| | | 5-35% basic species | 15 | 14 | 14 | 15 | 14 | 14 | 15 |
| CE-SDS (reducing) | % peak area | ≥90% sum of heavy chain + light chain. | 98 | 99 | 98 | 99 | 98 | 98 | 98 |
| CE-SES (non-reducing) | % peak area | ≥90% main peak. | 99 | 98 | 98 | 98 | 97 | 97 | 97 |
| | | ≤10.0% LMWS. | 0 | 1 | 0 | 0 | 0 | 0 | 1 |
| Potency | % | 60-150% potency relative to reference | 108 | 116 | 108 | 108 | 107 | 100 | 103 |
| | | standard | | | | | | | |
| Endotoxin | EU/mL | ≤32.00 EU/ml | <0.18 | NS | NS | NS | <0.18 | NS | <0.18 |
| Vial integrity | NA | Pass if all vials integral | Pass | NS | Pass | NS | Pass | NS | Pass |

NS: Not scheduled

TABLE 11
Long-term stability of exemplary CSL324 formulation after 18 months at 25° C. ± 3° C.

| | | | Number of months | | | | | |
|---|---|---|---|---|---|---|---|---|
| Test | Units | Provisional acceptance criteria | 0 | 3 | 6 | 9 | 12 | 18 |
| Particle count (LO) | No. of | ≤6,000 particles of ≥10 μm | 12 | 35 | 37 | 13 | 50 | 24 |
| average cumulative | particles/ | ≤600 particles of ≥25 μm | 1 | 1 | 2 | 0 | 3 | 1 |
| counts/container | container | | | | | | | |
| Description | N/A | Opalescent to clear, yellow to | Pass | Pass | Pass | Pass | Pass | Pass |
| | | colourless liquid. No visible particles. | | | | | | |
| pH | pH units | 5.5-5.9 | 5.6 | 5.6 | 5.6 | 5.6 | 5.6 | 5.7 |
| Protein concentration by | mg/mL | 110-130 mg/ml | 121 | 121 | 121 | 121 | 121 | 121 |
| UV spectrophotometry | | | | | | | | |
| (A280 nm) | | | | | | | | |
| SE-HPLC | % peak area | ≤5.0% HMWS | 0.7 | 2.9 | 3.5 | 3.7 | 4.3 | 4.7 |
| | | ≥95.0% monomer | 99.2 | 96.9 | 96.3 | 96.1 | 95.5 | 95.0 |
| | | 10-30% acidic species | 16 | 18 | 22 | 26 | 30 | 48 |
| CEX-HPLC | % peak area | 50-80% main peak | 69 | 66 | 61 | 57 | 55 | 36 |
| | | 5-35% basic species | 15 | 16 | 17 | 17 | 15 | 16 |
| CE-SDS (reducing) | % peak area | ≥90% sum of heavy chain + light chain. | 98 | 98 | 97 | 97 | 97 | 96 |
| CE-SES (non-reducing) | % peak area | ≥90% main peak. | 99 | 97 | 96 | 95 | 94 | 93 |
| | | ≤10.0% LMWS. | 0 | 1 | 2 | 2 | 3 | 4 |
| Potency | % | 60-150% potency relative to reference | 108 | 109 | 109 | 99 | 96 | 96 |
| | | standard | | | | | | |
| Endotoxin | EU/mL | ≤32.00 EU/ml | <0.18 | NS | NS | NS | <0.18 | NS |
| Vial integrity | NA | Pass if all vials integral | Pass | NS | Pass | NS | Pass | NS |

NS: Not scheduled

### Example 7: Toxicokinetics and Bioavailability of Subcutaneous Administration to Cynomolgus Monkeys

The goal of the following experiment was to assess the toxicokinetic profile of CSL324, in a 6-week (two doses) intravenous versus subcutaneous study in cynomolgus monkeys.

Three groups, each comprised of two male and two female cynomolgus monkeys, were administered 9.3 mg/kg or 93 mg/kg CSL324 subcutaneously (0.7 mL/kg) or 10 mg/kg intravenously (1.0 mL/kg). Table 12 below indicates the CSL324 formulation that was administered to each group.

TABLE 12
CSL324 formulations administered to cynomolgus monkeys

| Formulation | Study group | | |
|---|---|---|---|
| component | 9.3 mg/kg SC | 93 mg/kg SC | 10 mg/kg IV |
| CSL324 | 13.3 mg/mL | 133 mg/mL | 10 mg/mL |
| Buffer | histidine | 20 mM histidine | 20 mM histidine |
| | (pH 5.7) | (pH 5.7) | (pH 6.4) |
| Stabiliser(s) | proline | 100 mM proline | 140 mM NaCl |
| | arginine | 100 mM arginine | — |
| Polysorbate 80 | present | 0.03% (w/v) | 0.02% (w/v) |

Assessment of general toxicity was based on clinical observations, feces observations, body weights, and clinical (hematology) and anatomic pathology evaluations. Injection sites were evaluated by Draize irritation scoring (Draize et al., 1944). Complete necropsies were performed on all animals, with a recording of macroscopic abnormalities for all tissues. Organ weights and microscopic examinations were conducted as indicated.

Blood for toxicokinetic evaluation was collected at 0 (predose), 1, 4, and 8, 15, 22, 29, 36, and 43. Granulocyte Colony Stimulating Factor (G-CSF) levels were evaluated as a pharmacodynamics endpoint.

The administered dose solutions were stable for up to 6 hours at room temperature in dosing equipment and contained test item concentrations within the acceptance criteria of 90 to 110% of the nominal concentration for all dose levels.

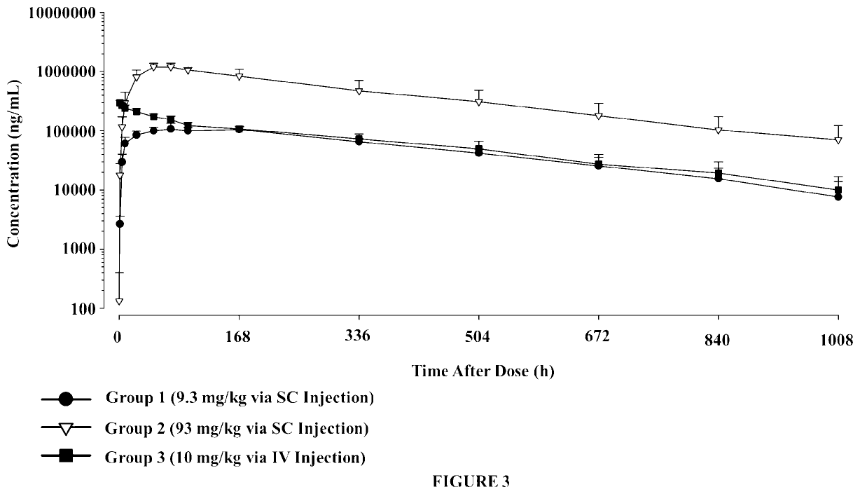

Table 13 shows the resulting toxicokinetic parameters in monkey serum of each group. Sex differences in CSL324 mean Cₘₐₓ and AUC₀₋ₜ values were less than 2-fold. Exposure, as assessed by CSL324 mean Cₘₐₓ and AUC₀₋ₜ values, generally increased with the increase in dose level from 9.3 to 93 mg/kg when administered via SC injection. The increases in mean Cₘₐₓ and AUC₀₋ₜ values were generally dose proportional. Following subcutaneous administration of 9.3 mg/kg, CSL324 was highly bioavailable, with a bioavailability value of 83.7% compared to 10 mg/kg IV injection. FIG. 3 shows mean (+SD) concentrations (ng/mL) of CSL324 in combined male and female monkey serum following a single dose via IV or SC injection.

TABLE 13
Mean CSL324 toxicokinetic parameters in monkey serum

| | Cₘₐₓ | Tₘₐₓ | AUC₀₋ₜ | t_{1/2} |
|---|---|---|---|---|
| Group | (μg/mL) | (h) | (h*μg/mL) | (h) |
| 9.3 mg/kg subcutaneous | 115 | 120 | 50400 | 182 |
| 93 mg/kg subcutaneous | 1250 | 60 | 414000 | 211 |
| 10 mg/kg intravenous | 298 | 1 | 64300 | 222 |

Numbers are from male and female monkeys combined. Median values are presented for Tₘₐₓ.

With the exception of one animal, all animals displayed detectable increases in serum G-CSF levels following CSL324 administration. However, G-CSF levels in animals who received 93 mg/kg of CSL324 were generally less than 2-fold higher on average than those G-CSF levels observed in animals who received 9.3 or 10 mg/kg of CSL324. Further, the occurrence of peak levels of G-CSF varied widely from as early as Day 2 to as late as Day 36 following CSL324 administration.

No CSL324-related effects were noted on survival, clinical observations, fecal observations, body weights, or hematology, and no macroscopic or microscopic changes were noted for the injection sites.

In conclusion, two doses (42 days apart) of 93 mg/kg CSL324 administered subcutaneously (in a formulation comprising histidine, proline, arginine and polysorbate, e.g., 20 mM histidine, pH 5.7, 100 mM proline, 100 mM arginine, and 0.03% polysorbate 80) to cynomolgus monkeys were well tolerated and did not elicit any adverse effects, with a bioavailability similar to intravenous administration. There was no evidence of irritation at the injection site following subcutaneous administration. The serum G-CSF levels increased post-CSL324 administration at both dose levels and routes. However, the peak levels and the timing thereof those peak levels of G-CSF varied widely, and there was a lack of consistent correlation between serum G-CSF and CSL324 levels.

The no observed adverse effect level (NOAEL) was considered to be 93 mg/kg by SC administration (AUC₀₋ₜ=414,000 h*μg/mL; Cmax=1,250 μg/mL for combined sexes) under the conditions of the study.

1. A liquid pharmaceutical formulation comprising a protein comprising an antigen binding domain that binds to or specifically binds to G-CSF receptor (G-CSFR), an organic acid buffer, a non-ionic surfactant and at least one amino acid stabiliser, wherein the formulation has a pH of 5.0 to 6.0.

2. The formulation of claim 1, wherein the protein is present in the formulation at a concentration of at least 20 mg/mL, at least 25 mg/mL, at least 50 mg/mL, or at least 100 mg/mL; or 110 mg/mL to 130 mg/mL.

3. (canceled)

4. (canceled)

5. The formulation of claim 1, wherein:
(a) the organic acid buffer is a histidine buffer; and/or
(b) the non-ionic surfactant is selected from the group consisting of polysorbate 80, polysorbate 20, and poloxamer 188; and/or
(c) the non-ionic surfactant is polysorbate 80; and/or
(d) the at least one amino acid stabiliser includes proline and/or arginine.

6. The formulation of claim 1, wherein:
(a) the organic acid buffer is present in the formulation at a concentration of 10 to 30 mM; and/or
(b) the non-ionic surfactant is present in the formulation at a concentration of 0.01% (w/v) to 0.05% (w/v); and/or
(c) the at least one amino acid stabiliser includes:
a. proline, wherein proline is present in the formulation at a concentration of 50 mM to 150 mM, and/or
b. arginine, wherein arginine is present in the formulation at a concentration of 50 mM to 150 mM.

7-12. (canceled)

13. The formulation of claim 1, wherein the formulation comprises a histidine buffer, proline, and polysorbate 80.

14. The formulation of claim 13, wherein the formulation further comprises arginine.

15. (canceled)

16. The formulation of claim 1, wherein the formulation has:
(a) a pH of 5.5 to 5.9 and comprises 12 mM to 30 mM histidine buffer, 0.02% to 0.04% (w/v) polysorbate 80, 60 mM to 125 mM proline, and mM to 125 mM arginine; and/or
(b) a pH of 5.5 to 5.9 and comprises 15 mM to 25 mM histidine buffer, to 0.04% (w/v) polysorbate 80, 90 mM to 110 mM proline, and 90 mM to 110 mM arginine; and/or
(c) a pH of 5.7 and comprises 20 mM histidine buffer, 0.03% (w/v) polysorbate 80, 100 mM proline, and 100 mM arginine.

17. (canceled)

18. (canceled)

19. The formulation of claim 1, wherein the formulation has:
(a) a dynamic viscosity of less than 20 mPa*s at 20° C., less than 10 mPa*s at 20° C., or less than 7 mPa*s at 20° C.; and/or
(b) an osmolality in the range of 250 mOsm/kg to 400 mOsm/kg.

20. (canceled)

21. The formulation of claim 1, wherein one or more or all of the following apply:
a) the formulation comprises no more than 5% high molecular weight species (HMWS), as determined by size exclusion high performance liquid chromatography (SE-HPLC);
b) at least 95% of the protein in the formulation is a monomer, as determined by SE-HPLC;
c) the formulation comprises no more than 50% acidic species, as determined by cation exchange high performance liquid chromatography (CEX-HPLC);
d) the formulation comprises no more than 20% basic species, as determined by cation exchange high performance liquid chromatography (CEX-HPLC); and
e) the formulation comprises no more than 5% low molecular weight species (LMWS), as determined by capillary electrophoresis with sodium dodecylsulfate (CE-SDS) under non-reducing conditions.

22. The formulation of claim 21, wherein the amount of HMWS, monomer, acidic species, basic species, or LMWS is determined after storage for a period of at least 1 month, at least 3 months, at least 6 months, at least 9 months, at least 12 months, at least 18 months, or at least 24 months at a temperature in the range of 2° C. to 30° C.

23. The formulation of claim 1, wherein the formulation has a volume in the range of 0.5 mL to 5 mL.

24. The formulation of claim 1, wherein the protein:
(a) inhibits granulocyte colony stimulating factor (G-CSF) signaling; and/or comprises an antigen binding domain of an antibody; and/or
(b) is selected from the group consisting of:
a. a single chain Fv fragment (scFv);
b. a dimeric scFv (di-scFv);
c. a diabody;
d. a triabody;
e. a tetrabody;
f. a Fab;
g. a F(ab′)2;
h. a Fv;
i. one of (a) to (h) is linked to a constant region of an antibody, Fc, or a heavy chain constant domain (C_{H}) C_{H}2 and/or C_{H}3; and
j. an antibody.

25. (canceled)

26. (canceled)

27. The formulation of claim 1, wherein the protein comprises:
(a) an antibody variable region comprising a heavy chain variable region (V_{H}) comprising an amino acid sequence set forth in SEQ ID NO: 4 and a light chain variable region (V_{L}) comprising an amino acid sequence set forth in SEQ ID NO: 5; and/or
(b) an antibody variable region comprising a V_{H} comprising three CDRs of a V_{H} comprising an amino acid sequence set forth in SEQ ID NO: 4 and a V_{L} comprising three CDRs of a V_{L} comprising an amino acid sequence set forth in SEQ ID NO: 5.

28. (canceled)

29. The formulation of claim 1, wherein the protein comprises an IgG₄ constant region, wherein the IgG₄ constant region is a stabilized IgG₄ constant region.

30. (canceled)

31. The formulation of claim 1, wherein the protein is an antibody comprising:
(i) a heavy chain comprising an amino acid sequence set forth in SEQ ID NO: 14 and a light chain comprising an amino acid sequence set forth in SEQ ID NO: 15; or
(ii) a heavy chain comprising an amino acid sequence set forth in SEQ ID NO: 18 and a light chain comprising an amino acid sequence set forth in SEQ ID NO: 15.

32. (canceled)

33. (canceled)

34. A method of reducing circulating neutrophils in a subject or treating or preventing a neutrophil-mediated condition in a subject, the method comprising administering the formulation of claim 1 to the subject, wherein the neutrophil-mediated condition is an autoimmune disease, an inflammatory disease, cancer, ischemia-reperfusion injury, or a neutrophilic dermatosis.

35-38. (canceled)

39. A prefilled syringe or autoinjector device comprising the pharmaceutical formulation of claim 1.

40. (canceled)

41. The method of claim 34, wherein the neutrophilic dermatosis is hidradenitis suppurativa.

42. The formulation of claim 27, wherein the formulation comprises at least 20 mg/ml of the protein.

43. The formulation of claim 42, wherein the formulation has a pH of 5.7 and comprises 20 mM histidine buffer, 0.03% (w/v) polysorbate 80, 100 mM proline, and 100 mM arginine.

44. The formulation of claim 27, wherein the formulation comprises 50 mg/ml of the protein.

45. The formulation of claim 44, wherein the formulation has a pH of 5.7 and comprises 20 mM histidine buffer, 0.03% (w/v) polysorbate 80, 100 mM proline, and 100 mM arginine.


SEQUENCE LISTING (18 sequences)

SEQ ID NO: 1 - protein, 319 aa, artificial
  amino acids 25-335 of Homo sapiens G-CSFR (hG-CSFR) with a C-terminal polyhistidine tag
ECGHISVSAPIVHLGDPITASCIIKQNCSHLDPEPQILWRLGAELQPGGRQQRLSDGTQESIITLPHLNHTQAFLSCALNWGNSLQILDQVELRAGYPPAIPHNLSCLMNLTTSSLICQWEPGPETHLPTSFTLKSFKSRGNCQTQGDSILDCVPKDGQSHCSIPRKHLLLYQNMGIWVQAENALGTSMSPQLCLDPMDVVKLEPPMLRTMDPSPEAAPPQAGCLQLSWEPWQPGLHINQKCELRHKPQRGEASWALVGPLPLEALQYELCGLLPATAYTLQIRCIRWPLPGHWSDWSPSLELRTTERAPTHHHHHHHH

SEQ ID NO: 2 - protein, 118 aa, artificial
  VH of C1.2
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAMLGELGWFDPWGQGTLVTVSS

SEQ ID NO: 3 - protein, 107 aa, artificial
  VL of C1.2
DIQMTQSPSALSASVGDRVTITCRASQGISSYLNWYQQKPGKAPKLLIYYASNLQNGIPSRFSGSGSGTDFTLTISSLQPEDFATYHCQQSYSTPLTFGGGTNVEIR

SEQ ID NO: 4 - protein, 118 aa, artificial
  VH of C1.2G
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSS

SEQ ID NO: 5 - protein, 107 aa, artificial
  VL  of C1.2G
DIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIK

SEQ ID NO: 6 - protein, 5 aa, artificial
  HCDR1 of C1.2
LYWMG

SEQ ID NO: 7 - protein, 17 aa, artificial
  C1.2 HCDR2
SISSSGGVTPYADSVKG

SEQ ID NO: 8 - protein, 9 aa, artificial
  C1.2 HCDR3
LGELGWFDP

SEQ ID NO: 9 - protein, 11 aa, artificial
  C1.2 LCDR1
RASQGISSYLN

SEQ ID NO: 10 - protein, 6 aa, artificial
  C1.2 LCDR2
ASNLQN

SEQ ID NO: 11 - protein, 9 aa, artificial
  C1.2 LCDR3
QQSYSTPLT

SEQ ID NO: 12 - protein, 8 aa, artificial
  consensus sequence of HCDR3 of C1.2
  VARIANT (5)..(5): X is an amino acid selected from the group consisting of tryptophan, glutamine, methionine, serine, phenylalanine, glutamic acid and histidine
  VARIANT (6)..(6): X iis an amino acid selected from the group consisting of phenylalanine, tyrosine, methionine, serine, glycine and isoleucine
  VARIANT (7)..(7): X is an amino acid selected from the group consisting of aspartic acid, methionine, glutamine, serine, leucine, valine, arginine and histidine
  VARIANT (8)..(8): X is an amino acid selected from the group consisting of proline gltuamic acid, alanine, leucine, phenylalanine, tyronis, threonine, asparagine, aspartic acid, serine , glycine, arginine, lysine
LGELXXXX

SEQ ID NO: 13 - protein, 9 aa, artificial
  consensus sequence of LCDR3 of C1.2
  VARIANT (1)..(1): X is an amino acid selected from the group consisting of glutamine, glutamic acid, histidine, alanine or serine
  VARIANT (2)..(2): X is an amino acid selected from the group consisting of glutamine, valine, phenylalanine, asparagine and glutamic acid
  VARIANT (3)..(3): X ian amino acid selected from the group consisting of serine or glycine
  VARIANT (4)..(4): X is an amino acid selected from the group consisting of tryptophan, methionine, phenylalanine, tyrosine, isoleucine and leucine
  VARIANT (5)..(5): X is an amino acid selected from the group consisting of glutamic acid, methionine, glutamine, tryptophan, serine, valine, asparagine, glycine, alanine, arganine, histidine, tyrosine, lysine or threonine
  VARIANT (6)..(6): X is an amino acid selected from the group consisting of tyrosine, methionine, isoleucine or threonine
  VARIANT (7)..(7): X is an amino acid selected from the group consisting of proline, alanine, histidine, glycine and lysine
  VARIANT (8)..(8): X is an amino acid selected from the group consisting of leucine, glutamine, methionine, alanine, phenylalanine, isoleucine, lysine, histidine and glycine
  VARIANT (9)..(9): X is an amino acid selected from the group consisting of threonine, phenylalanine, tyrosine, methionine, lysine, serine, histidine, proline, tryptophan, isoleucine, glutamine, glycine and valine
XXXXXXXXX

SEQ ID NO: 14 - protein, 445 aa, artificial
  C1.2G heavy chain IgG4 with S241P mutation
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 214 aa, artificial
  C1.2G with kappa light chain
DIQMTQSPSSLSASVGDRVTITCRASQGISSYLNWYQQKPGKAPKLLIYYASNLQNGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 836 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELHIILGLFGLLLLLTCLCGTAWLCCSPNRKNPLWPSVPDPAHSSLGSWVPTIMEEDAFQLPGLGTPPITKLTVLEEDEKKPVPWESHNSSETCGLPTLVQTYVLQGDPRAVSTQPQSQSGTSDQVLYGQLLGSPTSPGPGHYLRCDSTQPLLAGLTPSPKSYENLWFQASPLGTLVTPAPSQEDDCVFGPLLNFPLLQGIRVHGMEALGSF

SEQ ID NO: 17 - protein, 319 aa, artificial
  Ig and CRH domains of Macaca fascicularis G-CSFR (cynoG-CSFR) with a C-terminal polyhistidine tag
ECGHISVSAPIVHLGDPITASCIIKQNCSHLDLEPQILWRLGAELQPGGRQQRLSDGSQQSTITLPHLNHTRAFLSCALNWGNSLQILDQVELRAGYPPAVPRNLSCLMNLTTSSLICQWEPGPETHLPTSFTLKSFKSRGNCQTQGDSIMDCVPEDGQSHCSIPRRHLLLYQNMGIWVQAENALGTSMSPQLCLEPMDVVKLEPPMLRTMDPSPEAAPPQAGCLQLSWEPWQPALHINQKCELRHKPQSGEASWALVGPLPLEALRYELCGLLPATAYTLQIRCIRWPLPGHWSNWSPSLELRTTERAPTHHHHHHHH

SEQ ID NO: 18 - protein, 444 aa, artificial
  C1.2G heavy chain IgG4 with S241P mutation and lacking C-terminal lysine residue
EVQLLESGGGLVQPGGSLRLSCAASGFTFSLYWMGWVRQAPGKGLEWVSSISSSGGVTPYADSVKGRFTISRDNSKNTLYLQMNSLRAEDTAVYYCAKLGELGWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG